(19) United States
(12) Patent Application Publication
(10) Pub. No.: US 2005/0239872 A1
(43) Pub. Date: Oct. 27, 2005

(54) 1,5-distributed pyrrolid-2-one derivatives for use as ep4 receptor agonists in the teatment of eye diseases such as glaucoma

(76) Inventors: Xavier Billot, Montreal (CA); Robert N. Young, Senneville, CA (US); Yongxin Han, Kirkland, CA (US)

Correspondence Address: MERCK AND CO., INC, P O BOX 2000, RAHWAY, NJ, 07065-0907, US

(21) Appl. No.: 10/511,736

(22) PCT Filed: Jun. 2, 2003
(86) PCT No.: PCT/CA03/00838

§ 371 Date: Oct. 18, 2004

Related U.S. Application Data
(60) Provisional application No. 60/386,499, filed on Jun. 6, 2002.
(60) Provisional application No. 60/460,134, filed on Apr. 3, 2003.

(51) Int. Cl. A61K 31/4015

(52) U.S. Cl. 514/425

(57) ABSTRACT

This invention relates to potent selective agonists of the EP₄ subtype of prostaglandin E2 receptors of formula (1), their use or a formulation thereof in the treatment of glaucoma and other conditions which are related to elevated intraocular pressure in the eye of the patient.

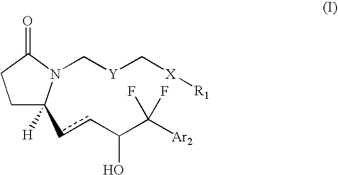

This case claims the benefit of provisional application U.S. Ser. No. 60/386,499, filed Jun. 6, 2002 and U.S. Ser. No. 60/460,134, filed Apr. 3, 2003.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

Many of the drugs formerly used to treat glaucoma proved unsatisfactory. Early methods of treating glaucoma employed pilocarpine and produced undesirable local effects that made this drug, though valuable, unsatisfactory as a first line drug. More recently, clinicians have noted that many O-adrenergic antagonists are effective in reducing intraocular pressure. While many of these agents are effective for this purpose, there exist some patients with whom this treatment is not effective or not sufficiently effective. Many of these agents also have other characteristics, e.g., membrane stabilizing activity, that become more apparent with increased doses and render them unacceptable for chronic ocular use and can also cause cardiovascular effects.

Agents referred to as carbonic anhydrase inhibitors decrease the formation of aqueous humor by inhibiting the enzyme carbonic anhydrase. While such carbonic anhydrase inhibitors are now used to treat elevated intraocular pressure by systemic and topical routes, current therapies using these agents, particularly those using systemic routes are still not without undesirable effects. Topically effective carbonic anhydrase inhibitors are disclosed in U.S. Pat. Nos. 4,386,098; 4,416,890; 4,426,388; 4,668,697; 4,863,922; 4,797,413; 5,378,703, 5,240,923 and 5,153,192.

Prostaglandins and prostaglandin derivatives are also known to lower intraocular pressure. There are several prostaglandin types, including the A, B, C, D, E, F, G, I and J-Series (EP 0561073 A1). U.S. Pat. No. 4,883,819 to Bito describes the use and synthesis of PGAs, PGBs and PGCs in reducing intraocular pressure. U.S. Pat. No. 4,824,857 to Goh et al. describes the use and synthesis of PGD₂ and derivatives thereof in lowering intraocular pressure including derivatives wherein C-10 is replaced with nitrogen. U.S. Pat. No. 5,001,153 to Ueno et al. describes the use and synthesis of 13,14-dihydro-15-keto prostaglandins and prostaglandin derivatives to lower intraocular pressure. U.S. Pat. No. 4,599,353 describes the use of eicosanoids and eicosanoid derivatives including prostaglandins and prostaglandin inhibitors in lowering intraocular pressure. See also WO 00/38667, WO 99/32441, WO 99/02165, WO 00/38663, WO 01/46140, EP 0855389, JP 2000-1472, U.S. Pat. No. 6,043,275 and WO 00/38690.

Prostaglandin and prostaglandin derivatives are known to lower intraocular pressure by increasing uveoscleral outflow. This is true for both the F type and A type of prostaglandins. This invention is particularly interested in those compounds that lower IOP via the uveoscleral outflow pathway and other mechanisms by which the E series prostaglandins (PGE2) may facilitate IOP reduction. The four recognized subtypes of the EP receptor are believed to modulate the effect of lowering IOP (EP1, EP2, EP3 and EP4; J. Lipid Mediators Cell Signaling, Vol. 14, pages 83-87 (1996)). See also J. Ocular Pharmacology, Vol. 4, 1, pages 13-18 (1988); J. Ocular Pharmacology and Therapeutics, Vol. 11, 3, pages 447-454 (1995); J. Lipid Mediators, Vol. 6, pages 545-553 (1993); U.S. Pat. Nos. 5,698,598 and 5,462,968 and Investigative Ophthalmology and Visual Science, Vol. 31, 12, pages 2560-2567 (1990). Of particular interest to this invention are compounds, which are agonist of the EP4 subtype receptor.

A problem with using prostaglandins or derivatives thereof to lower intraocular pressure is that these compounds often induce an initial increase in intraocular pressure, can change the color of eye pigmentation and cause proliferation of some tissues surrounding the eye.

As can be seen, there are several current therapies for treating glaucoma and elevated intraocular pressure, but the efficacy and the side effect profiles of these agents are not ideal. Therefore, there still exist the need for new and effective therapies with little or no side effects.

A variety of disorders in humans and other mammals involve or are associated with abnormal or excessive bone loss. Such disorders include, but are not limited to, osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

Two different types of cells called osteoblasts and osteoclasts are involved in the bone formation and resorption processes, respectively. See H. Fleisch, Bisphosphonates In Bone Disease, From The Laboratory To The Patient, 3rd Edition, Parthenon Publishing (1997), which is incorporated by reference herein in its entirety. Osteoblasts are cells that are located on the bone surface. These cells secrete an osseous organic matrix, which then calcifies. Substances such as fluoride, parathyroid hormone, and certain cytokines such as protaglandins are known to provide a stimulatory effect on osetoblast cells. However, an aim of current research is to develop therapeutic agents that will selectively increase or stimulate the bone formation activity of the osteoblasts.

Osteoclasts are usually large multinucleated cells that are situated either on the surface of the cortical or trabecular bone or within the cortical bone. The osteoclasts resorb bone in a closed, sealed-off microenvironment located between the cell and the bone. The recruitment and activity of osteoclasts is known to be influenced by a series of cytokines and hormones. It is well known that bisphosphonates are selective inhibitors of osteoclastic bone resorption, making these compounds important therapeutic agents in the treatment or prevention of a variety of systemic or localized bone disorders caused by or associated with abnormal bone resorption. However, despite the utility of bisphosphonates there remains the desire amongst researchers to develop additional therapeutic agents for inhibiting the bone resorption activity of osteoclasts.

Prostaglandins such as the PGE₂ series are known to stimulate bone formation and increase bone mass in mammals, including man. It is believed that the four different receptor subtypes, designated EP₁, EP₂, EP₃, and EP₄ are involved in mediating the bone modeling and remodeling processes of the osteoblasts and osteoclasts. The major prostaglandin receptor in bone is EP₄, which is believed to provide its effect by signaling via cyclic AMP.

In present invention it is further found that the formula I agonists of the EP₄ subtype receptor are useful for stimulating bone formation. WO 02/24647, WO 02/42268, EP 1132086, EP 855389, EP 1114816, WO 01/46140 and WO 01/72268 disclose EP₄ agonists. However, they do not disclose the compounds of the instant invention.

### SUMMARY OF THE INVENTION

This invention relates to potent selective agonists of the EP₄ subtype of prostaglandin E2 receptors, their use or a formulation thereof in the treatment of glaucoma and other conditions that are related to elevated intraocular pressure in the eye of a patient. Another aspect of this invention relates to the use of such compounds to provide a neuroprotective effect to the eye of mammalian species, particularly humans. This invention further relates to the use of the compounds of this invention for mediating the bone modeling and remodeling processes of the osteoblasts and osteoclasts.

More particularly, this invention relates to the use of EP4 agonist having the structural formula I to treat ocular hypertension and/or glaucoma: 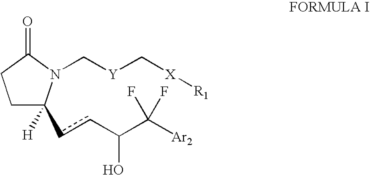
or a pharmaceutically acceptable salt, enantiomer, diastereomer, prodrug or mixture thereof, wherein,
X is (CH₂)ₙ, O or S;
Y represents (C(Rb)₂)ₙ, triple bond, 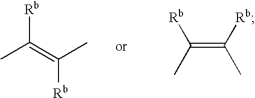
R₁ represents hydroxy, CN, CHO, NHSO₂R₆, CONHSO₂R₆, CON(R₆)₂ hydroxymethylketone, (CH₂)ₚCO₂R₆, (CH₂)ₙSO₃R₆, C₁₋₄ alkoxy, or (CH₂)ₙC₅₋₁₀heterocyclyl, said heterocyclyl unsubstituted or substituted with 1 to 3 groups of Rₐ and optionally containing an acidic hydroxyl group, with the proviso that when X is a bond R₁ is not (CH₂)ₚCO₂R₆, C₁₋₄ alkoxy, —(CH₂)ₙNR₆R₇, CHO, NHSO₂R₆, CONHSO₂R₆, CON(R₆)₂, or hydroxymethylketone;
R² and R³ independently represents hydrogen, or C₁₋₄ alkyl;
R₆ and R₇ independently represents hydrogen, or C₁₋₆ alkyl, C₃₋₁₀ cyclcoalkyl, (CH₂)ₚC₆₋₁₀aryl, (CH₂)ₚC₅₋₁₀heterocyclyl, CR²R³OC(O)OC₃₋₁₀cycloalkyl or CR²R³OC(O)O C₁₋₁₀oalkyl;
Ar₂ independently represent (CH₂)ₘC₆₋₁₀aryl, (CH₂)ₘC₅₋₁₀heteroaryl, (CH₂)ₘC₃₋₁₀heterocycloalkyl, (CH₂)ₘC₃₋₈ cycloalkyl said cycloalkyl, heterocycloalkyl, aryl or heteroaryl unsubstituted or substituted with 1-3 groups of Rₐ;
Rₐ represents C₁₋₆ alkoxy, C₁₋₆ alkyl, CF₃, nitro, amino, cyano, C₁₋₆ alkylamino, or halogen;
R^{b} independently represents H, halogen, C₁₋₆ alkyl, C₃₋₆ cylcoalkyl or 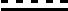
   represents a double or single bond;
p represents 1-3;
n represents 04; and
m represents 0-8.

This and other aspects of the invention will be realized upon inspection of the invention as a whole.

### DETAILED DESCRIPTION OF THE INVENTION

The invention is described herein in detail using the terms defined below unless otherwise specified.

The term “therapeutically effective amount”, as used herein, means that amount of the EP₄ receptor subtype agonist of formula I, or other actives of the present invention, that will elicit the desired therapeutic effect or response or provide the desired benefit when administered in accordance with the desired treatment regimen. A preferred therapeutically effective amount relating to the treatment of abnormal bone resorption is a bone formation, stimulating amount. Likewise, a preferred therapeutically effective amount relating to the treatment of ocular hypertension or glaucoma is an amount effective for reducing intraocular pressure and/or treating ocular hypertension and/or glaucoma.

“Pharmaceutically acceptable” as used herein, means generally suitable for administration to a mammal, including humans, from a toxicity or safety standpoint.

The term “prodrug” refers to compounds which are drug precursors which, following administration and absorption, release the claimed drug in vivo via some metabolic process. A non-limiting example of a prodrug of the compounds of this invention would be an acid of the pyrrolidinone group, where the acid functionality has a structure that makes it easily hydrolyzed after administration to a patient. Exemplary prodrugs include acetic acid derivatives that are non-narcotic, analgesics/non-steroidal, anti-inflammatory drugs having a free CH₂COOH group (which can optionally be in the form of a pharmaceutically acceptable salt, e.g. —CH₂COO—Na+), typically attached to a ring system, preferably to an aromatic or heteroaromatic ring system.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, without alternating or resonating double bonds between carbon atoms. It may contain from 1 to 4 rings, which are fused. Examples of cycloalkyl groups are cyclopropyl, cyclobutyl, cyclopentyl, cyclohexyl, and cycloheptyl.

Alkoxy refers to C₁-C₆ alkyl-O—, with the alkyl group optionally substituted as described herein. Examples of alkoxy groups are methoxy, ethoxy, propoxy, butoxy and isomeric groups thereof.

Halogen (halo) refers to chlorine, fluorine, iodine or bromine.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. The preferred aryl groups are phenyl, naphthyl and phenanthrenyl. Aryl groups may likewise be substituted as defined. Preferred substituted aryls include phenyl and naphthyl.

The term “heterocycloalkyl” refers to a cycloalkyl group (nonaromatic) having 3 to 10 carbon atoms in which one of the carbon atoms in the ring is replaced by a heteroatom selected from O, S or N, and in which up to three additional carbon atoms may be replaced by hetero atoms.

The term “cycloalkyl” refers to a cyclic alkyl group (nonaromatic) having 3 to 10 carbon atoms.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of this type are pyrrole, pyridine, oxazole, thiazole, tetrazole, and oxazine. For purposes of this invention the tetrazole includes all tautomeric forms. Additional nitrogen atoms may be present together with the first nitrogen and oxygen or sulfur, giving, e.g., thiadiazole.

The term heterocyclyl or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

For purposes of this invention, heterocyclyls containing acidic hydroxyl groups are those heterocyclyl groups that have an acidic hydroxy atom and can have a pKa in the range of 3 to 7. Non-limiting examples of heterocyclyls containing acidic hydroxyl groups are: 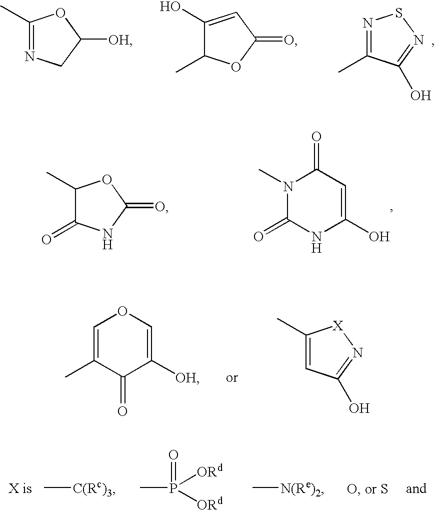
each R^{c} independently is H, fluorine,
cyano or C₁₋₄ alkyl;
each R^{d} independently is H, C₁₋₄ alkyl,
or a pharmaceutically acceptable cation;
each R^{e} independently is H, —C(═O)—R^{f},
or —SO₂R^{e}, wherein R^{f} is C₁₋₄ linear alkyl
or phenyl

The term “agonist” as used herein means EP₄ subtype compounds of formula I interact with the EP₄ receptor to produce maximal, super maximal or submaximal effects compared to the natural agonist, PGE2. See Goodman and Gilman, The Pharmacological Basis of Therapeutics, 9^{th} edition, 1996, chapter 2.

One embodiment of this invention is realized when compounds as described below are used to treat ocular hypertension and/or glaucoma. In particular, the compounds are those wherein R₁ is CN, (CH₂)ₙC₅₋₁₀heterocyclyl, (CH₂)ₚCO₂R₆ or (CH₂)ₙSO₃R₆, said heterocyclyl unsubstituted or substituted with 1 to 3 groups of Rₐ and all other variables are as originally described. A subembodiment of this invention is realized when X is (CH₂)ₙ,. Another subembodiment of this invention is when X is Sulfur. Still another sub-embodiment of this invention is when X is oxygen. When R₁ is (CH₂)ₚCO₂R₆, and X is sulfur, the sulfur is hexavalent.

Another embodiment of this invention is realized when Y is (CH₂)ₙ and all other variables are as originally described.

Still another embodiment of this invention is realized when Y is C(halo)₂ and all other variables are as originally described.

Still another embodiment of this invention is realized when Y is a double bond as described by 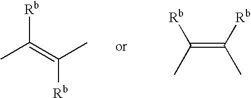
and all other variables are as originally described.

Still another embodiment of this invention is realized when Y is a triple bond and all other variables are as originally described.

Another embodiment of this invention is realized when R₁ is (CH₂)ₙC₅₋₁₀heterocyclyl, said heterocyclyl unsubstituted or substituted with 1 to 3 groups of Rₐ and all other variables are as originally described. A subembodiment of this invention is realized when X is (CH₂)ₙ, and Y is (CH₂)ₙ. Still another subembodiment of this invention is realized when when X is (CH₂)ₙ, and Y is C(halo)₂. Yet another subembodiment of this invention is realized when X is a bond and Y is a double bond as described by 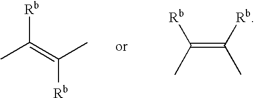
Another subembodiment of this invention is realized when X is S.

Another embodiment of this invention is realized when R₁ is (CH₂)ₚCO₂R₆, and all other variables are as originally described. A sub-embodiment of this invention is realized when X is (CH₂)ₙ, and Y is (CH₂)ₙ.

Another embodiment of this invention is realized when Ar₂ is (CH₂)ₘC₆₋₁₀aryl, said aryl unsubstituted or substituted with 1 to 3 groups of Rₐ and all other variables are as originally described.

Still another embodiment of this invention is realized when R₁ is a tetrazole unsubstituted or substituted with an Rₐ group and all other variables are as originally described. A subembodiment of this invention is realized when X is (CH₂)ₙ, and Y is (CH₂)ₙ, C(halo)₂ or a double bond as described by 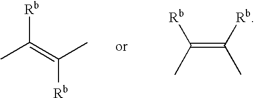
Another subembodiment of this invention is realized when X is S.

Still another embodiment of this invention is realized when Ar₂ is a phenyl unsubstituted or substituted with 1 to 3 groups of Rₐ and all other variables are as originally described.

Yet another embodiment of this invention is realized when R₁ is tetrazolyl and Ar₂ is phenyl, said tetrazolyl unsubstituted or substituted with a Rₐ group and phenyl is unsubstituted or substituted with 1-3 groups of Rₐ, and all other variables are as originally described. A subembodiment of this invention is realized when X is (CH₂)ₙ, and Y is (CH₂)ₙ, C(halo)₂ or a double bond as described by 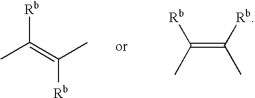
Another subembodiment of this invention is realized when X is sulfur.

Still another embodiment of this invention is realized when a compound of formula II: 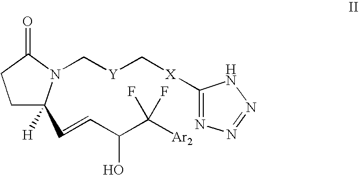
or a pharmaceutically acceptable salt, enantiomer, diastereomer, pro drug or mixture thereof is used, wherein X, Y, Ar₂ and n are as previously described.

A subembodiment of this invention is realized when X is (CH₂)ₙ, and Y is (CH₂)ₙ, C(halo)₂ or a double bond as described by 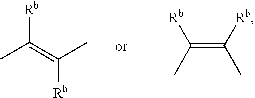
and Ar₂ is phenyl. Another subembodiment of this invention is realized when n is 4.

A subembodiment of this invention is realized when X is S.

Compounds used in this invention are:
(5R)-5-[(1E)-3-hydroxy-4,4-difluoro-4-phenylbut-1-enyl]-1-{4-[(1-methyl-1H-tetrazol-5-yl)]butyl}pyrrolidin-2-one,
4{(2R)-2-[(1E)-3-hydroxy-4,4-difluoro-4-phenylbut-1-enyl]-5-oxopyrrolidin-1-yl}butyl cyanate,
3-[4-{(2R)-2-[(1E)-3-hydroxy-4,4-difluoro-4phenylbut-1-enyl]-5-oxopyrrolidin-1-yl}butyl)]propanoic acid,
[4-{(2R)-2-[(1E)-3-hydroxy-4,4-difluoro-4-phenylbut-1-enyl]-5-oxopyrrolidin-1-yl}butyl)]methanesulfonic acid,
(5R)-5-[(1E)-3-hydroxy-4,4-difluoro-4-phenylbut-1-enyl]-1-{4-[1H-tetrazol-5-ylmethyl)butyl}-pyrrolidin-2-one,
[4-{(2R)-2-[(1E)-3-hydroxy-4,4-difluoro-4-phenylbut-1-enyl]-5-oxopyrrolidin-1-yl}butyl)]acetic acid,
(5R)-5-[(1E)-3-hydroxy-4,4-difluoro-4-phenylbut-1-enyl]-1-{4-[(1-methyl-1H-tetrazol-5-yl)thio]butyl]pyrrolidin-2-one,
(5R)-5-[(1E)-3-hydroxy-4,4-difluoro-4-phenylbut-1-enyl]-1-[4-(1H-tetrazol-5-ylthio)butyl]pyrrolidin-2-one,
3-[4-{(2R)-2-[(1E)-3-hydroxy-4,4-difluoro-4-phenylbut-1-enyl]-5-oxopyrrolidin-1-yl}butyl)thio]propanoic acid,
[4-{(2R)-2-[(1E)-3-hydroxy-4,4-difluoro-4-phenylbut-1-enyl]-5-oxopyrrolidin-1-yl}butyl)thio]methanesulfonic acid,
(5R)-5-[(1E)-3-hydroxy-4,4-difluoro-4-phenylbut-1-enyl]-1-[4-(methylsulfonyl)butyl]-pyrrolidin-2-one,
[4-(2R)-2-[(1E)-3-hydroxy-4,4-difluoro-4-phenylbut-1-enyl]-5-oxopyrrolidin-1-yl}butyl)thio]acetic acid,
(5R)-5[(1E)-4,4-difluoro-3-hydroxy-4-phenylbut-1-enyl]-1-[6-(1H-tetrazol-5-yl)hexyl]pyrrolidin-2-one,
7-{(2R)-2-[(1E,3R)-4,4-difluoro-3-hydroxy-4-phenylbut-1-enyl]-5-oxopyrrolidin-1-yl}heptanoic acid,
isopropyl 7-{(2R)-2-[(1E,3R)-4,4-difluoro-3-hydroxy-4-phenylbut-1-enyl]-5-oxopyrrolidin-1-yl}heptanoate,
7-{(2S)-2-[(3R)-4,4-difluoro-3-hydroxy-4-phenylbutyl]-5-oxopyrrolidin-1-yl}heptanoic acid,
(5Z)-7-{(2R)-2-[(1E,3R)-4,4-difluoro-3-hydroxy-4-phenylbut-1-enyl]-5-oxopyrrolidin-1-yl}hept-5-enoic acid,
isopropyl (5Z)-7-{(2R)-2-[(1E,3R)-4,4-difluoro-3-hydroxy-4-phenylbut-1-enyl]-5-oxopyrrolidin-1-yl}hept-5-enoate,
7-{(2R)-2-[(1E,3R (3-chlorophenyl)-4,4-fluoro-3-hydroxybut-1-enyl]-5-oxopymolidin-1-yl}heptanoic acid,
isopropyl 7-{(2R)-2-[(1E,3R)-4-(3-chlorophenyl)-4,4-difluoro-3-hydroxybut-1-enyl]-5-oxopyrrolidin-1-yl}heptanoate,
7-((2R)-2-{(1E,3R)-4,4-difluoro-3-hydroxy t[3-trifluoromethyl)phenyl]but-1-enyl}-5-oxopyrrolidin-1-yl)heptanoic acid,
isopropyl 7-((2R-2-{(1E,3R)-4,4-difluoro-3-hydroxy-4-[3-(trifluoromethyl)phenyl]but-1-enyl}-5-oxopytrolidin-1-yl)heptanoate,
cyclopentyl 7-{(2R)-2-[(1E,3R)-4,4-difluoro-3-hydroxy-4-phenylbut-1-enyl]-5-oxopyrrolidin-1-yl}heptanoate,
7-{(2R)-2-[(1E,3R)-4,4-fluoro-3-hydroxy-3-methyl-4-phenylbut-1-enyl]-5-oxopyrrolidin-1-yl}heptanoic acid,
isopropyl 7-{(2R)-2-[(1E,3R)-4,4-difluoro-3-hydroxy-3-methyl-4-phenylbut-1-enyl]-5-oxopyrrolidin-1-yl}heptanoate,
isobutyl 7-{(2R)-2-[(1E,3R)-4,4-difluoro-3-hydroxy-4-phenylbut-1-enyl]-5-oxopyrrolidin-1-yl}heptanoate, and
cyclohexyl 7-{(2R)-2-[(1E,3R)-4,4-difluoro-3-hydroxy-4-phenylbut-1-enyl]-5-oxopyrrolidin-1-yl}heptanoate, or a pharmaceutically acceptable salt, enantiomer, diastereomer, prodrug, or mixture thereof.

Additional compounds are:
(5R)-5-[(1E)-(3R)-hydroxy-4,4-difluoro-4-phenylbut-1-enyl]-1-{4-[(1-methyl-1H-tetrazol-5-yl)]butyl}pyrrolidin-2-one,
4-{(2R)-2-[(1E)-(3R)-hydroxy-4,4-difluoro-4-phenylbut-1-enyl]-5-oxopyrrolidin-1-yl}butyl cyanate,
3-[4-{(2R)-2-[(1E)-(3R)-hydroxy-4,4-difluoro-4-phenylbut-1-enyl]-5-oxopyrrolidin-1-yl}butyl)]propanoic acid,
[4-{(2R)-2-[(1E)-(3R)-hydroxy-4,4-difluorophenylbut-1-enyl]-1-oxopyrrolidin-1-yl}butyl)]methanesulfonic acid,
(5R)-5-[(1E)-(3R)-hydroxy-4,4-difluoro-4-phenylbut-1-enyl]-1-{4-[1H-tetrazol-5-ylmethyl)butyl}pyrrolidin-2-one,
[4-{(2R)-2-[(1E)-(3R)-hydroxy-4,4-difluorophenylbut-1-enyl]-5-oxopyrrolidin-1-yl}butyl)]acetic acid,
(5R)-5-[(1E)-(3R)-hydroxy-4,4-difluoro-4-phenylbut-1-enyl]-1-{4-[(1-methyl-1H-tetrazol-5-yl)thio]butyl}pyrrolidin-2-one,
(5R)-5-[(1E)-(3R)-hydroxy-4,4-difluoro-4-phenylbut-1-enyl]-1-[4(1H-tetrazol-5-ylthio)butyl]pyrrolidin-2-one,
3-[4{(2R)-2-[(1E)-(3R)-hydroxy-4,4-difluoro-4-phenylbut-1-enyl]-5-oxopyrrolidin-1-yl}butyl)thio]propanoic acid,
[4-{(2R)-2-[(1E)-(3R)-hydroxy-4,4-difluoro-4-phenylbut-1-enyl]-5-oxopyrrolidin-1-yl}butyl)thio]methanesulfonic acid,
(5R)-5-[(1E)-(3R)-hydroxy-4,4-difluorophenylbut-1-enyl]-1-[4 (methylsulfonyl)butyl]-pyrrolidin-2-one,
[4-{(2R)-2-[(1E)-(3R)-hydroxy-4,4-difluoro-4-phenylbut-1-enyl]-5-oxopyrrolidin-1-yl}butyl)thio]acetic acid, or
(5R)-5[(1E)-4,4-difluoro-(3R)-hydroxy-4-phenylbut-1-enyl]-1-[6-(1H-tetrazol-5-yl)hexyl]pyrrolidin-2-one, a pharmaceutically acceptable salt, enantiomer, diastereomer, prodrug, or mixture thereof.

Another embodiment of this invention is directed to a composition containing an EP₄ agonist of Formula I and optionally a pharmaceutically acceptable carrier.

Yet another embodiment of this invention is directed to a method for decreasing elevated intraocular pressure or treating glaucoma by administration, preferably topical or intra-camaral administration, of a composition containing an EP₄ agonist of Formula I and optionally a pharmaceutically acceptable carrier. Use of the compounds of formula I for the manufacture of a medicament for treating elevated intraocular pressure or glaucoma or a combination thereof is also included in this invention.

This invention is further concerned with a process for making a pharmaceutical composition comprising a compound of formula I.

This invention is further concerned with a process for making a pharmaceutical composition comprising a compound of formula I, and a pharmaceutically acceptable carrier.

The claimed compounds bind strongly and act on PGE₂ receptor, particularly on the EP₄ subtype receptor and therefore are useful for preventing and/or treating glaucoma and ocular hypertension.

Dry eye is a common ocular surface disease afflicting millions of people. Although it appears that dry eye may result from a number of unrelated pathogenic causes, the common end result is the breakdown of the tear film, which results in dehydration of the exposed outer surface of the eye. (Lemp, Report of the Nation Eye Institute/Industry Workshop on Clinical Trials in Dry Eyes, The CLAO Joumel, 21(4):221-231 (1995)). One cause for dry eye is the decreased mucin production by the conjunctival cells and/or corneal epithelial cells of mucin, which protects and lubricates the ocular surface (Gipson and Inatomi, Mucin genes expressed by ocular surface epithelium. Progress in Retinal and Eye Research, 16:81-98 (1997)). Functional EP4 receptors have been found in human conjuctival epithelial cells (see U.S. Pat. No. 6,344,477, incorporated by reference in its entirey) and it is appreciated that both human corneal epithelial cells (Progess in Retinal and Eye Research, 16:81-98(1997)) and conjuctival cells (Dartt et al. Localization of nerves adjacent to goblet cells in rat conjucntiva. Current Eye Research, 14:993-1000 (1995)) are capable of secreting mucins. Thus, the compounds of formula I are useful for treating dry eye.

Macular edema is swelling within the retina within the critically important central visual zone at the posterior pole of the eye. An accumulation of fluid within the retina tends to detach the neural elements from one another and from their local blood supply, creating a dormancy of visual function in the area. It is believed that EP4 agonist which lower IOP are useful for treating diseases of the macular such as macular edema or macular degeneration. Thus, another aspect of this invention is a method for treating macular edema or macular degeneration.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, January 1994, pp. 37-44; Investigative Ophthamol. & Visual Science, 32, 5, Apr. 1991, pp. 1593-99. It is believed that EP₄ agonist which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, or increasing retinal and optic nerve oxygen tension or providing a neuroprotective effect or a combination thereof by using an EP₄ agonist of formula I.

The compounds produced in the present invention are readily combined with suitable and known pharmaceutically acceptable excipients to produce compositions which may be administered to mammals, including humans, to achieve effective IOP lowering. Thus, this invention is also concerned with a method of treating ocular hypertension or glaucoma by administering to a patient in need thereof one of the compounds of formula I alone or in combination with a β-adrenergic blocking agent such as timolol, betaxolol, levobetaxolol, carteolol, levobunolol, a parasympathomimetic agent such as pilocarpine, a sympathomimetic agents such as epinephrine, iopidine, brimonidine, clonidine, para-aminoclonidine, a carbonic anhydrase inhibitor such as dorzolamide, acetazolamide, metazolamide or brinzolamide; a Maxi-K channel blocker as disclosed in U.S. Ser. No. 60/389,205, filed Jun. 17, 2002 (Attorney Docket 21121PV), No. 60/389,222, filed Jun. 17, 2002 (Attorney docket 21092PV), No. 60/458,981, filed Mar. 27, 2003 (Attorney docket 21101PV4), No. 60/424,790, filed Nov. 8, 2002 (Attorney docket 21260PV), No. 60/424,808, filed Nov. 8, 2002 (Attorney docket 21281PV), Ser. No. 09/765,716, filed Jan. 17, 2001, Ser. No. 09/64738, filed Jan. 17, 2001 and PCT publications WO 02/077168 and WO 02/02060863, all incorporated by reference in their entirety herein, a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine.

This invention is also concerned with a method for increasing retinal and optic nerve head blood velocity, or increasing retinal and optic nerve oxygen tension or providing a neuroprotective effect or a combination thereof by administering to a patient in need thereof one of the compounds of formula I alone or in combination with a β-adrenergic blocking agent such as timolol, betaxolol, levobetaxolol, carteolol, levobunolol, a parasympathomnimetic agent such as pilocarpine, a sympathomimetic agents such as epinephrine, iopidine, brimonidine, clonidine, para-aminoclonidine, a carbonic anhydrase inhibitor such as dorzolamide, acetazolamide, metazolamide or brinzolamide; a Maxi-K channel blocker as disclosed in U.S. Ser. No. 60/389,205, filed Jun. 17, 2002 (Attorney Docket 21121PV), No. 60/389,222, filed Jun. 17, 2002 (Attorney docket 21092PV), No. 60/458,981, filed Mar. 27, 2003 (Attorney docket 21101PV4), No. 60/424,790, filed Nov. 8, 2002 (Attorney docket 21260PV), No. 60/424,808, filed Nov. 8, 2002 (Attorney docket 21281PV), Ser. No. 09/765,716, filed Jan. 17, 2001, Ser. No. 09/764,738, filed Jan. 17, 2001 and PCT publications WO 02/077168 and WO 02/02060863, all incorporated by reference in their entirety herein, a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine. Use of the compounds of formula I for the manufacture of a medicament for increasing retinal and optic nerve head blood velocity, or increasing retinal and optic nerve oxygen tension or providing a neuroprotective effect or a combination thereof is also included in this invention.

This invention is further concerned with a method for treating macular edema or macular degeneration by administering to a patient in need thereof one of the compounds of formula I alone or in combination with a O-adrenergic blocking agent such as timolol, betaxolol, levobetaxolol, carteolol, levobunolol, a parasympathomimetic agent such as pilocarpine, a sympathomimetic agents such as epinephrine, iopidine, brimonidine, clonidine, para-aminoclonidine, a carbonic anhydrase inhibitor such as dorzolamide, acetazolamide, metazolamide or brinzolamide; a Maxi-K channel blocker as disclosed in U.S. Ser. No. 60/389,205, filed Jun. 17, 2002 (Attorney Docket 21121PV), No. 60/389,222, filed Jun. 17, 2002 (Attorney docket 21092PV), No. 60/458,981, filed Mar. 27, 2003 (Attorney docket 21101PV4), No. 60/424,790, filed Nov. 8, 2002 (Attorney docket 21260PV), No. 60/424,808, filed Nov. 8, 2002 (Attorney docket 21281PV), Ser. No. 09/765,716, filed Jan. 17, 2001, Ser. No. 09/764,738, filed Jan. 17, 2001 and PCT publications WO 02/077168 and WO 02/02060863, all incorporated by reference in their entirety herein, a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-3-chloro-6-methoxy-indazol-1-yl)-1-methyl-ethylamine. Use of the compounds of formula I for the manufacture of a medicament for macular edema or macular degeneration is also included in this invention.

The EP₄ agonist used in the instant invention can be administered in a therapeutically effective amount intravaneously, subcutaneously, topically, transdermally, parenterally or any other method known to those skilled in the art. Ophthalmic pharmaceutical compositions are preferably adapted for topical administration to the eye in the form of solutions, suspensions, ointments, creams or as a solid insert. Ophthalmic formulations of this compound may contain from 0.001 to 5% and especially 0.001 to 0.1% of medicament. Higher dosages as, for example, up to about 10% or lower dosages can be employed provided the dose is effective in reducing intraocular pressure, treating glaucoma, increasing blood flow velocity or oxygen tension. For a single dose, from between 0.001 to 5.0 mg, preferably 0.005 to 2.0 mg, and especially 0.005 to 1.0 mg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-ceUulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats, rabbits and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mammalian eye will be from once up to three times daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The compounds of the instant invention are also useful for mediating the bone modeling and remodeling processes of the osteoblasts and osteoclasts. See PCT US99/23757 filed Oct. 12, 1999 and incorporated herein by reference in its entirety. The major prostaglandin receptor in bone is EP₄, which is believed to provide its effect by signaling via cyclic AMP. See Ikeda T, Miyaura C, Ichikawa A, Narumiya S, Yoshiki S and Suda T 1995, In situ localization of three subtypes (EP₁, EP₃ and EP₄) of prostaglandin E receptors in embryonic and newborn mice., J Bone Miner Res 10 (sup 1):S 172, which is incorporated by reference herein in its entirety. Use of the compounds of formula I for the manufacture of a medicament for mediating the bone modeling and remodeling processes are also included in this invention.

Thus, another object of the present invention is to provide methods for stimulating bone formation, i.e. osteogenesis, in a mammal comprising administering to a mammal in need thereof a therapeutically effective amount of an EP₄ receptor subtype agonist of formula I.

Still another object of the present invention to provide methods for stimulating bone formation in a mammal in need thereof comprising administering to said mammal a therapeutically effective amount of an EP₄ receptor subtype agonist of formula I and a bisphosphonate active. Use of the compounds of formula I for the manufacture of a medicament for stimulating bone formation is also included in this invention.

Yet another object of the present invention to provide pharmaceutical compositions comprising a therapeutically effective amount of an EP₄ receptor subtype agonist of formula I and a bisphosphonate active.

It is another object of the present invention to provide methods for treating or reducing the risk of contracting a disease state or condition related to abnormal bone resorption in a mammal in need of such treatment or prevention, comprising administering to said mammal a therapeutically effective amount of an EP₄ receptor subtype agonist of formula I. Use of the compounds of formula I for the manufacture of a medicament for treating or reducing the risk of contracting a disease state or condition related to abnormal bone resorption is also included in this invention.

The disease states or conditions related to abnormal bone resorption include, but are not limited to, osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy, and multiple myeloma.

Within the method comprising administering a therapeutically effective amount of an EP₄ receptor subtype agonist of formula I and a bisphosphonate active, both concurrent and sequential administration of the EP₄ receptor subtype agonist of formula I and the bisphosphonate active are deemed within the scope of the present invention. Generally, the formulations are prepared containing 5 or 10 mg of a bisphosphonate active, on a bisphosphonic acid active basis. With sequential administration, the agonist and the bisphosphonate can be administered in either order. In a subclass of sequential administration the agonist and bisphosphonate are typically administered within the same 24 hour period. In yet a further subclass, the agonist and bisphosphonate are typically administered within about 4 hours of each other.

Nonlimiting examples of bisphosphonate actives useful herein include the following:
Alendronic acid, 4-amino-1-hydroxybutylidene-1,1-bisphosphonic acid;
Alendronate (also known as alendronate sodium or alendronate monosodium trihydrate), 4-amino-1-hydroxybutylidene-1,1-bisphosphonic acid monosodium trihydrate;
Alendronic acid and alendronate are described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety;
Cycloheptylaminomethylene-1,1-bisphosphonic acid, YM 175, Yamanouchi (cimadronate), as described in U.S. Pat. No. 4,970,335, to Isomura et al., issued Nov. 13, 1990, which is incorporated by reference herein in its entirety;
1,1-dichloromethylene-1,1-diphosphonic acid (clodronic acid), and the disodium salt (clodronate, Procter and Gamble), are described in Belgium Patent 672,205 (1966) and J. Org. Chen 32, 4111 (1967), both of which are incorporated by reference herein in their entirety;
1-hydroxy-3-(1-pyrrolidinyl)-propylidene-1,1-bisphosphonic acid (EB-1053);
1-hydroxyethane-1,1-diphosphonic acid (etidronic acid);
1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety;
6-amino-1-hydroxyhexylidene-1,1-bisphosphonic acid (neridronate);
3-(dimethylamino)-1-hydroxypropylidene-1,1-bisphosphonic acid (olpadronate);
3-amino-1-hydroxypropylidene-1,1-bisphosphonic acid (pamidronate);
[2-(2-pyridinyl)ethylidene]-1,1-bisphosphonic acid (piridronate) is described in U.S. Pat. No. 4,761,406, which is incorporated by reference in its entirety,
1-hydroxy-2-(3-pyridinyl)-ethylidene-1,1-bisphosphonic acid (risedronate);
(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety; and
1-hydroxy-2-(1H-imidazol-1-yl)ethylidene-1,1-bisphosphonic acid (zolendronate).

A non-limiting class of bisphosphonate actives useful in the instant invention are selected from the group consisting of alendronate, cimadronate, clodronate, tiludronate, etidronate, ibandronate, neridronate, olpandronate, risedronate, piridronate, pamidronate, zolendronate, pharmaceutically acceptable salts thereof, and mixtures thereof.

A non-limiting subclass of the above-mentioned class in the instant case is selected from the group consisting of alendronate, pharmaceutically acceptable salts thereof, and mixtures thereof.

A non-limiting example of the subclass is alendronate monosodium trihydrate.

In the present invention, as it relates to bone stimulation, the agonist is typically administered for a sufficient period of time until the desired therapeutic effect is achieved. The term “until the desired therapeutic effect is achieved”, as used herein, means that the therapeutic agent or agents are continuously administered, according to the dosing schedule chosen, up to the time that the clinical or medical effect sought for the disease or condition being mediated is observed by the clinician or researcher. For methods of treatment of the present invention, the compounds are continuously administered until the desired change in bone mass or structure is observed. In such instances, achieving an increase in bone mass or a replacement of abnormal bone structure with normal bone structure are the desired objectives. For methods of reducing the risk of a disease state or condition, the compounds are continuously administered for as long as necessary to prevent the undesired condition. In such instances, maintenance of bone mass density is often the objective.

Nonmiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

The instant compounds are also useful in combination with known agents useful for treating or preventing bone loss, bone fractures, osteoporosis, glucocorticoid induced osteoporosis, Paget's disease, abnormally increased bone turnover, periodontal disease, tooth loss, osteoarthritis, rheumatoid arthritis,, periprosthetic osteolysis, osteogenesis imperfecta, metastatic bone disease, hypercalcemia of malignancy, and multiple myeloma. Combinations of the presently disclosed compounds with other agents useful in treating or preventing osteoporosis or other bone disorders are within the scope of the invention. A person of ordinary skill in the art would be able to discern which combinations of agents would be useful based on the particular characteristics of the drugs and the disease involved. Such agents include the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; and the pharmaceutically acceptable salts and mixtures thereof. A preferred combination is a compound of the present invention and an organic bisphosphonate. Another preferred combination is a compound of the present invention and an estrogen receptor modulator. Another preferred combination is a compound of the present invention and an estrogen. Another preferred combination is a compound of the present invention and an androgen receptor modulator. Another preferred combination is a compound of the present invention and an osteoblast anabolic agent.

Regarding treatment of abnormal bone resorption and ocular disorders, the formula I agonists generally have an EC₅₀ value from about 0.001 nM to about 100 microM, although agonists with activities outside this range can be useful depending upon the dosage and route of administration. In a subclass of the present invention, the agonists have an EC₅₀ value of from about 0.01 microM to about 10 microM. In a further subclass of the present invention, the agonists have an EC₅₀ value of from about 0.1 microM to about 10 microM. EC₅₀ is a common measure of agonist activity well known to those of ordinary skill in the art and is defined as the concentration or dose of an agonist that is needed to produce half, i.e. 50%, of the maximal effect. See also, Goodman and Gilman's, The Pharmacologic Basis of Therapeutics, 9th edition, 1996, chapter 2, E. M. Ross, Phanracodynamics, Mechanisms of Drug Action and the Relationship Between Drug Concentration and Effect, and PCT US99/23757, filed Oct. 12, 1999, which are incoroporated by reference herein in their entirety.

The herein examples illustrate but do not limit the claimed invention. Each of the claimed compounds are EP4 agonists and are useful for a number of physiological ocular and bone disorders.

The compounds of this invention can be made, with some modification, in accordance with U.S. Pat. No. 6,043,275, EP0855389, U.S. Ser. No. 60/337,228 (Merck Docket No. MC052) and WO 01/46140, all of which are incorporated herein by reference in their entirety. The following non-limiting examples, given by way of illustration, is demonstrative of the present invention.

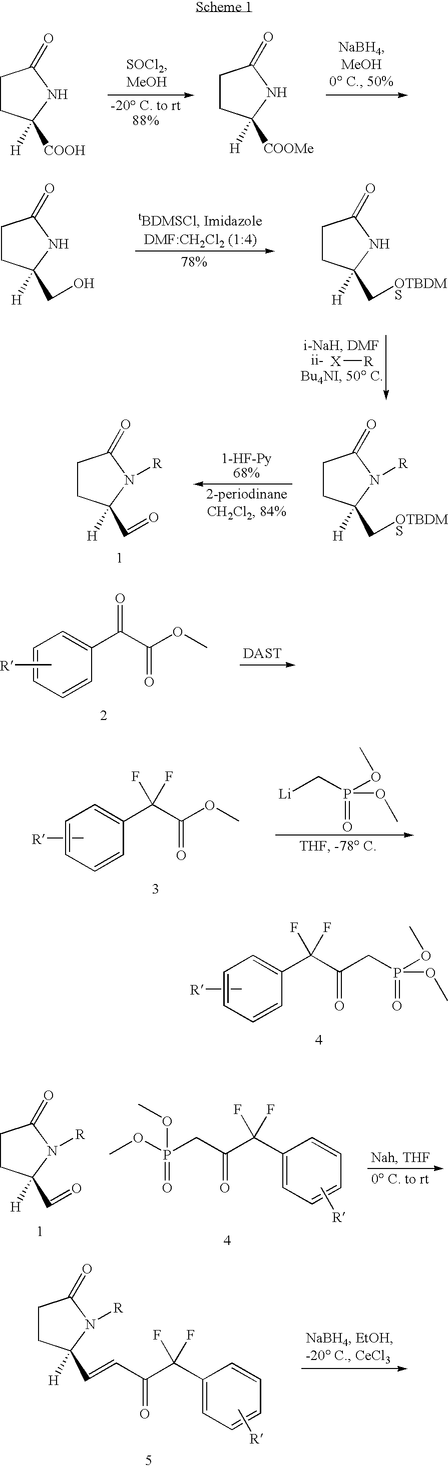

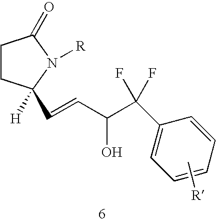

The compounds embodied in this application may be synthesized in part by Scheme 1. Pyroglutamic acid is converted to the corresponding ester through the action of a dehydrating agent such as thienylchloride and an alcohol such as methanol. The ester can be reduced by a reducing agent such as sodium borohydrate to provide the corresponding alcohol. Protection of the alcohol with a suitable protecting group such as t-butyldimethylsilylchloride in the presence of a base such as imidazole provides these silyl-protected alcohol. Reacting the protected alcohol amide with a strong base such as sodium hydride and thence with an alkyl halide (RX) provides the N-allylated product. Alternatively, a protecting group such as paramethoxybenzylbromide may be substituted for RX to provide a protected analog of the N-alkylated product. Removal of the silyl-protecting group with a reagent such as HF-pyridine and then subsequent oxidation of the free alcohol with an oxidizing agent such as 2-periodinane then provides the aldehyde 1.

An aryl ketoester such as 2 is reacted with a fluoronating agent such as DAST to provide the difluoro ester 3. Reaction with lithiomethylene dimethoxyphosphonate provides the ketophosphonate 4. Reaction of the aldehyde 1 with the ketophosphonate 4 in the presence of a strong base such as sodium hydride provides the olefin 5. Reduction of the ketone group with a reducing agent such as sodium borohydride provides the alcohol 6. The alcohol 6 may subsequently be protected with a suitable protecting group such as t-butyldimethylsilylchloride and thence, if desired, the protected amide (such as paramethoxybenzyl=R) it would be deprotected utilizing an oxidizing agent such as ceric ammonium nitrate. The free amide then be reacted with a strong base (sodium hydride) and then reacted with a suitably elaborated alkyl halide RX to provide the final products.

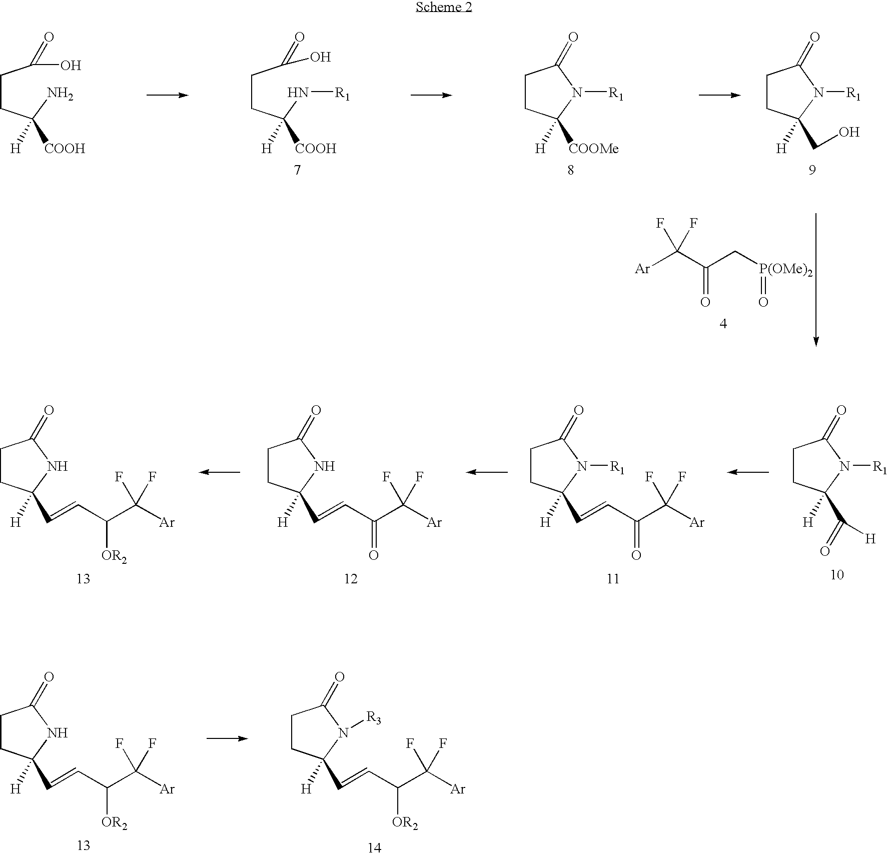

An alternative scheme is shown in Scheme 2. Glutamic acid is treated with an aromatic aldehyde such as paramethoxybenzyldehyde, in the presence of reducing agents such as sodium borohydride or sodium cyanoborohydride to provide the N-alkylated product 7. Cyclization and ester synthesis are accomplished using methanol and acidic catalysts to afford 8. Reduction of the ester to the alcohol 9 is accomplished using a reducing agent such as sodium borohydride and then oxidation of the alcohol moiety with common oxidation reagents such as pyridine, sulfur trioxide, etc. provides the aldehyde 10. Reaction of the aldehyde 10 with difluoroketophosphonate 4 in the presence of a strong base such as LDA or sodium hydride allows the formation of enone 11. The protecting group R₁ can then be removed in an oxidation step using a reagent such as cerium ammonium nitrate to afford the lactam 12. The enone moiety is reduced to the allylalcohol using a common reducing agent such as sodium borohydride and then protected in the usual manner by reaction with t-butyldimethylsilylchloride to afford a protected alcohol 13. The amide 13 can then be alkylated with a variety of groups R₃ by reaction first with a strong base such as sodium hydride in a polar non protic solvent in the presence of a phase-transfer catalyst such as tetrabutylammoniumiodide and then addition of electrophilic reagent R₃X where X is the halogen or a suitable leaving group to provide 14.

If R₃ is an aromatic group, the reaction conditions are that compound 13 is mixed with a base such as cesium carbonate in dioxane, the aromatic R₃X where X is halogen or triflate and a catalytic amount of palladium catalyst and the mixture is heated.

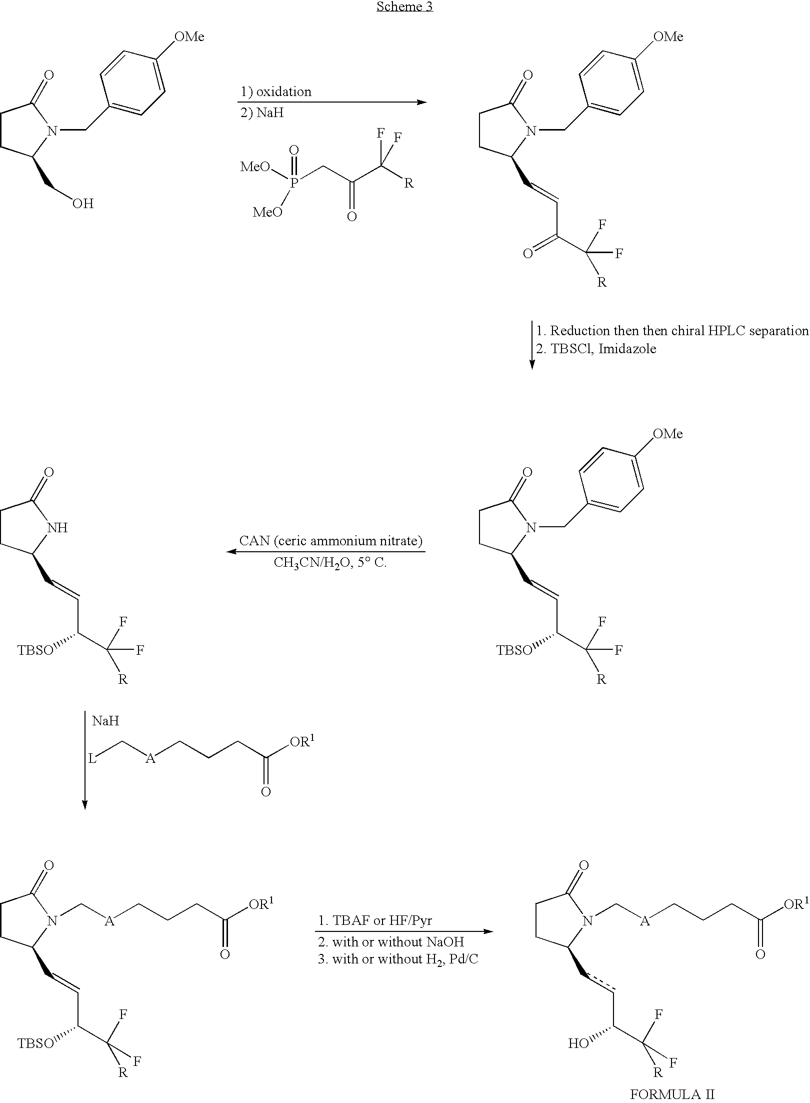

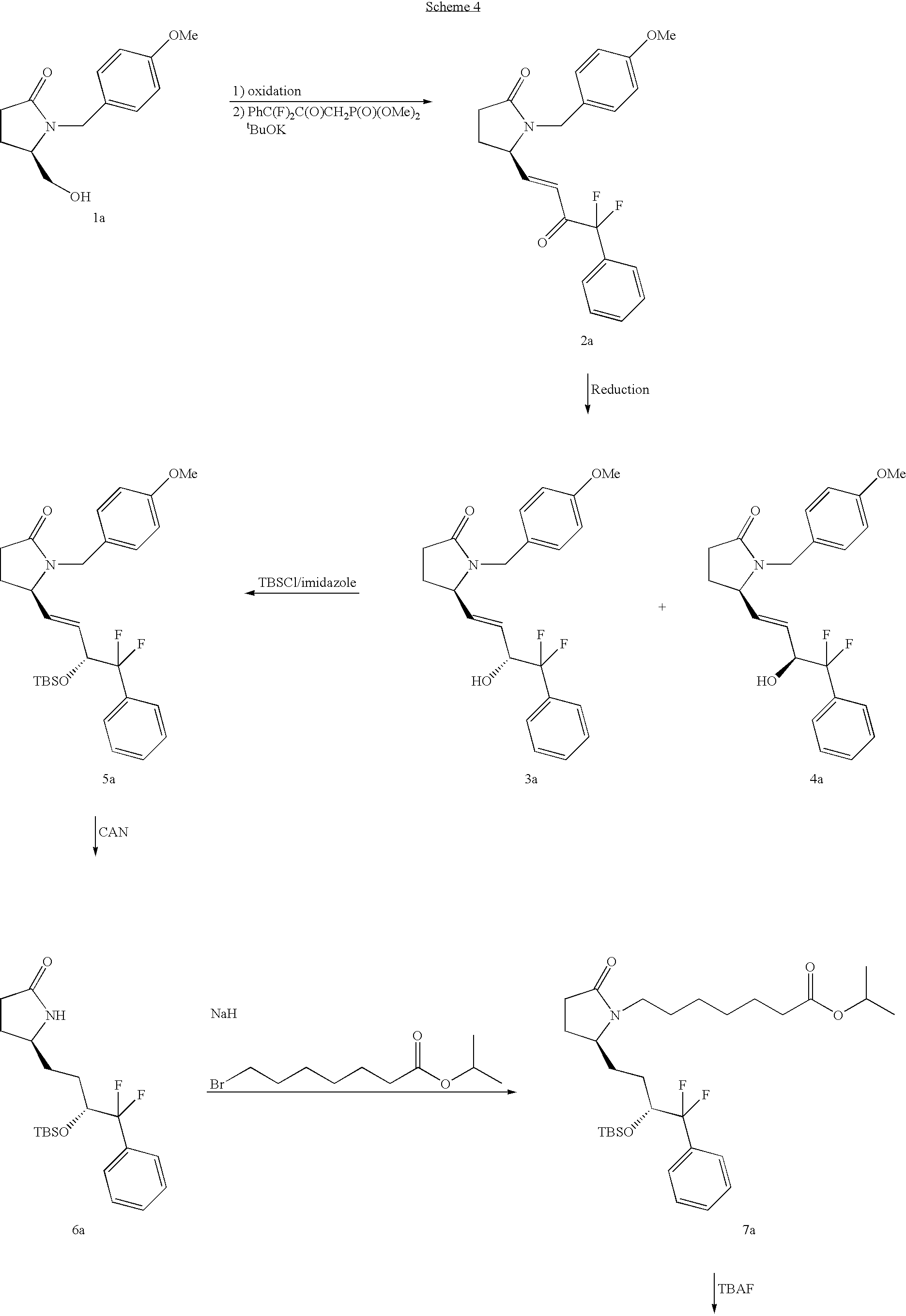

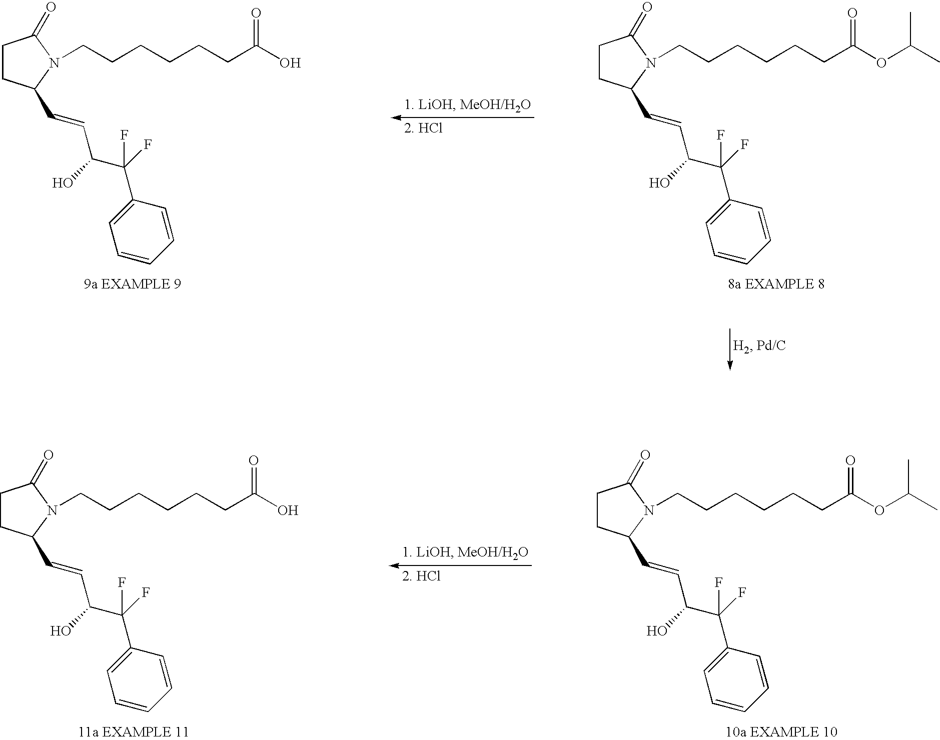

### EXAMPLES

### Preparation 1

### 7-(2R-formyl]-5-oxo-pyrrolidin-1-yl)-heptanenitrile

### Step A: 7-[2R-(tert-Butyl-dimethyl-silanyloxymethyl)-5-oxo-pyrrolidin-1-yl]-heptanenitrile

To a mixture of NaH (60% in oil, 3.836 g, 0.0959 mol, washed with 25 mL DMF) in DMF (250 mL) was added a solution of 5R-(tert-butyl-dimethyl silanyloxymethyl)-pyrrolidin-2-one (Tetrahdedron: Asymmetry, 1996, 7, 2113) (20.00 g, 87.19 mmol) in DMF (50 mL). The reaction was stirred at room temperature for 1.5 h and a solution of 7-bromoheptanonitrile (16.574 g, 87.19 mmol) in DMF (50 mL) was added. The reaction was stirred at 90° C. for 3 h. The reaction was cooled to room temperature and water (750 mL) was added. The aqueous solution was washed with EtOAc (4×250 mL). The combined organic solutions were washed with water (2×250 mL), dried (MgSO₄), filtered, and concentrated. Purification by medium pressure chromatography eluting with a solvent gradient (9:1 hexanes:EtOAc to 7:3 hexanes:EtOAo to 1:1 hexanes:EtOAc) provided 7-[2R-(tert-butyl-dimethyl silanyloxymethyl)-5-oxo-pyrrolidin-1-yl)-heptanenitrile (22.46 g). ¹H N (CDCl₃) δ 3.69-3.55 (m, 4H), 2.99 (m, 1H), 2.42 (m, 1H), 2.34-2.24 (m, 3H), 2.05 (m, 1H), 1.81 (m, 1H), 1.67-1.42 (m, 6H), 1.31 (m, 2H), 0.86 (s, 9H), 0.03 (s, 6H); MS 339.3 (M+1).

### Step B: 7-(2R-Hydroxymethyl-5-oxo-pyrrolidin-1-yl-heptanenitrile

A solution of tetrabutylammonium fluoride (1M in THF, 100.0 mL, 100.0 mmol) was slowly added to a solution of 7-[2R-(tert-butyl-dimethyl-silanyloxymethyl)-5-oxo-pyrrolidin-1-yl]-heptanenitrile (22.39 g, 66.13 mmol) in THF (400 mL) at 0° C. The reaction was warmed to room temperature and was stirred for 4 h. Saturated aqueous NaHCO₃ (250 mL) was added and the volatiles were removed in vacuo. The remaining aqueous solution was washed with CHCl₃ (4×200 mL). The combined organic solutions were dried (MgSO₄), filtered, and concentrated. Purification by medium pressure chromatography eluting with a solvent gradient (9:1 hexanes:EtOAc to 4:1 hexanes:EtOAc to 7:3 hexanes:EtOAc to 6:4 hexanes:EtOAc to 1:1 hexanes:EtOAc to EtOAc to 9:1 EtOAc:MeOH) provided 7-(2R-hydroxymethyl-5-oxo-pyrrolidin-1-yl)-heptanenitrile (14.922 g). ¹H NMR (CDCl₃) δ 3.78 (dd, 1H), 3.71-3.58 (m, 3H), 3.00 (m, 1H), 2.46 (m, 1H), 2.36-2.27 (m, 3H), 2.08 (m, 1H), 1.93 (m, 1H), 1.77 (m, 1H), 1.68-1.43 (m, 6H), 1.32 (m, 2H); MS 225.1 (M+1).

### Step C: 7-(2R-formyl-5-oxo-pirolidin-1-yl)-heptanenitrile

To a solution of 7-(2R-hydroxymethyl-5-oxo-pyrrolidin-1-yl)-heptanenitrile (336 mg, 1.5 mmole) in CH₂Cl₂ (7 ml) was added portion-wise Dess-Martin periodinane (636 mg, 1.5 mmole) and the reaction mixture was stirred 1 h at rt. Solvent is removed in vacuo, and the residue is triturated with toluene, filtered on celite and solvent removed to give 7-2R-formyl-5-oxo-pyrrolidin-1-yl)-heptanenitrile as an oil.

### Preparation 2

### Dimethyl 3,3-difluoro-2-oxo-3-phenyl-propylphosphonate

To a solution of dimethyl methanephosphonate (1.139 g, 9.18 mmole) in 20 ml TBF is added dropwise nBuLi (1.6 M in hexanes, 5.73 ml, 9.18 mmole) at −78° C. This solution is stirred 30 min at −78° C. and then added to a solution of 2,2-difluorophenylacetic acid methyl ester (1.75 g, 8.74 mmole) at −78° C. The reaction mixture is allowed to reach rt and then acetic acid (1.5 ml) and 10 ml water are added. The aqueous phase is extracted three times with 30 ml AcOEt, the organic phases are then washed with water, brine, dried on Na₂SO₄ and the solvent is removed under reduced pressure. Purification of the residual oil by silica gel flash chromatography (3:7 Acetone:toluene) to give Dimethyl 3,3-difluoro-2-oxo-3-phenyl-propylphosphonate as an oil. ¹H NMR (CDCl₃) δ 7.65-7.40 (m, 5H), 3.71-3.58 (m, 3H), 3.77 (s, 3H), 3.74 (s, 3H), 3.35 (d, J=22 Hz, 2H); MS 279.1 (M+1).

### Preparation 3

### (5R)-1-(4-chlorobutyl)-5-[(1E)-3-hydroxy-4-phenylbut-1-enyl]-pyrrolidin-2-one

### Step A: (5R)-(tert-butyl-dimethyl-silanyloxymethyl)-1-(4-chlorobutyl)pyrrolidin-2-one

To a solution of (5R)-(tert-butyl-dimethyl-silanyloxymethyl)-pyrrolidin-2-one (Tetrahedron: Asymmetry, 1996, 7,2113) (2.83 g, 12.34 mmol) in 60 ml DMF was added NaH (95%, 325.7 mg, 13.57 mmol) in one portion and the mixture was heated at 50° C. for 30 min. Then 4-bromo-1-chlorobutane (2.96 g, 17.27 mmol) and a catalytic amount of nBu₄NI were added and the mixture was stirred at 50° C. for 1 h. The reaction was cooled to room temperature and water (100 ml) was added. The aqueous phase was extracted with AcOEt (4×200 ml), the organic phases were washed with water (200 ml), brine (100 ml), dried on MgSO₄, filtered and the solvent was removed under reduced pressure. The residual oil was purified by flash column-chromatography on silica gel (eluent AcOEt 1: Hexanes 1) to provide (5R)-(tert-butyl-dimethyl-silanyloxymethyl)-1-(4-chlorobutyl)pyrrolidin-2-one as an oil. ¹H NMR (CDCl₃) 3.71-3.53 (m, 6), 3.05 (m, 1H), 2.46-2.24 (m, 2H), 2.05 (m, 1H), 1.84-1.61 (m, 4H), 0.85 (s, 9H), 0.03 (s, 6H); MS 320.2-322.2 (M+1).

### Step B: (5R)-1-(4-chlorobutyl)-5-(hydroxymethyl)pyrrolidin-2-one

To a solution of (5R)-(tert-butyl-dimethyl-silanyloxymethyl)-1-(4-chlorobutyl)pyrrolidin-2-one (1.95 g, 6.11 mmol) in CH₂Cl₂ (25 ml) in a Teflon Erlenmeyer at 0° C. was added dropwise HF-pyridine complex (1 ml), and the solution was allowed to reach room temperature, and was stirred for 1.5 h. Water (20 ml) and 1N HCl (1 ml) were added to the reaction mixture. The aqueous phase was extracted with CH₂Cl₂ (4×30 ml), the organic phases was washed with brine (20 ml), dried on MgSO₄, filtered and the solvent was removed under reduced pressure. The residual oil was purified by flash column-chromatography on silica gel (eluent Acetone 1: Toluene 1) to provide (5R)-1-(4-chlorobutyl)₅-(hydroxymethyl)pyrrolidin-2-one as an oil. ¹H NMR (CDCl₃) 4.00 (s, 1H), 3.71-3.53 (m, 6H), 3.03 (m, 1H), 2.46-2.22 (m, 2H), 2.14-1.88 (m, 2H), 1.79-1.55 (m, 4H); MS 206.1-208.1 (M+1).

### Step C: (2R)-1-(4-chlorobutyl)-5-oxopgrrolidine-2-carboxaldehyde

To a solution of (5R)-1-(4-chlorobutyl)-5-(hydroxymethyl)pyrrolidin-2-one (309.6 mg, 1.5 mmol) in CH₂Cl₂ (7 ml) was added Dess-Martin periodinane (638 mg, 1.5 mmol) portionwise over 40 min at room temperature. After 1 h, the solvent was removed under reduced pressure, and the residue triturated with Et₂O (3×5 ml), filtered on a celite plug, and the solvent removed. (2R)-1-(4-chlorobutyl)-5-oxopyrrolidine-2-carboxaldehyde was obtained as a colorless oil. ¹H NMR (CDCl₃) 9.58 (s, 1H), 4.18 (m, 1H), 3.65 (m, 1H), 3.53 (t, J=8 Hz, 2), 3.08 (m, 1H), 2.43 (m, 2H), 2.30(m, 1H), 2.08 (m, 1H), 1.78-1.56 (m, 4).

### Step D: (5R)-1-(4-chlorobutyl)-5-[(1E3-oxo-4-phenylbut-1-enyl]pyrrolidin-2-one

To a solution of (3-phenyl-2-oxo-propyl)-phosphonic acid dimethyl ester (938 mg, 4 mmol) in DME (20 ml) at 0° C. was added portionwise NaH 95% (100.8 mg, 4.2 mmol), and the mixture was stirred 20 min at 0° C. Then a solution of (2R)-1-(4-chlorobutyl)-5-oxopyrrolidine-2-carboxaldehyde in DME (5 ml) was added dropwise and the reaction mixture was allowed to reach room temperature, and stirred overnight. A half-saturated solution of NH₄Cl (10 ml) was added and the aqueous phase was extracted with AcOEt (4×60 ml); the organic phases was washed with water (20 ml), brine (20 ml), dried on MgSO₄, filtered and the solvent was removed under reduced pressure. The residual oil was purified by flash column-chromatography on silica gel (eluent Acetone 2: Toluene 8) to provide (5R)-1-(4-chlorobutyl)-5-[(1E)-3oxo-4-phenylbut-1-enyl]pyrrolidin-2-one as an oil. ¹H NMR (CDCl₃) 7.35-7.20 (m, 5H), 6.64 (dd, J=15.7 Hz, 8.2 Hz, 1H), 6.25 (d, J=15.7 Hz, 111), 4.17 (m, 1H), 3.85 (s, 2H), 3.55-3.50 (m, 3H), 2.77 (m, 1H), 2.43-2.17 (m, 3H), 1.81-1.75 (m, 1H), 1.70-1.51 (m, 4H).

### Step E: (5R)-1-(4-chlorobutyl)-5-[(1E)-3-hydroxy-4-phenylbut-1-enyl]pyrrolidin-2-one

To a solution of (5R)-1-(4-chlorobutyl)-5-[(1E)-3-oxo-4-phenylbut-1-enyl]pyrrolidin-2-one (161 mg, 0.50 mmol) in MeOH (5 ml) at −20° C. was added portionwise NaBH₄ (31 mg, 0.8 mmol). The mixture was stirred at −20° C. for 1 h, and the solvent was removed under reduced pressure. The residue was dissolved in a mixture of water (5 ml) and 1N HCl (1 ml), the aqueous phase was extracted with AcOEt (3×15 ml); the organic phases was washed with water (5 ml), brine (5 ml), dried on MgSO₄, filtered and the solvent was removed under reduced pressure. The residual oil was purified by flash column-chromatography on silica gel (eluent Acetone 4: Toluene 6) to provide both diastereoisomers of (5R)-1-(4-chlorobutyl)-5-[(1E)-3-hydroxy-4-phenylbut-1-enyl]pyrrolidin-2-one as an oil. ¹H NMR (CDCl₃) 7.36-7.22 (m, 5H), 5.78 (m, 1H), 5.51 (m, 1H), 4.44 (m, 1H), 4.07 (m, 1H), 3.59-3.45 (m, 3H), 2.95-2.77 (m, 3H), 2.44-2.19 (m, 3H), 2.43-2.17 (m, 3H), 1.70-1.55 (m, 51).

### Preparation 4

### (5R)-5-[(1E)-3-hydroxy-4-phenylbut-1-enyl]-1-4{-[(triisopropylsilyl)thio]butyl}-pyrrolidin-2-one

To a solution of (5R)-1-(4-chlorobutyl)-5-[(1E)-3-hydroxy-4-phenylbut-1-enyl]pyirolidin-2-one (273.9 mg, 0.852 mmol) in THF (5 ml) were added triisopropylsilylsulfide (324.4 mg, 1.70 mmol), a catalytic amount of nBu₄NI and portionwise NaH 95% (30.7 mg, 1.28 mmol). The mixture was heated to 50° C. for 1 h. The reaction was cooled to room temperature and water (2 ml) was added. The aqueous phase was extracted with AcOEt (4×10 ml), the organic phases were washed with water (2 ml), brine (2 ml), dried on MgSO₄, filtered and the solvent was removed under reduced pressure. The residual oil was purified by flash column-chromatography on silica gel (eluent AcOEt 1: Hexanes 3) to provide both diastereoisomers of (5R)-5-[(1E)-3-hydroxy-4-phenylbut-1-enyl]-1-{4-[(triisopropylsilyl)thio]butyl}pyrrolidin-2-one as an oil. ¹H NMR (CDCl₃) 7.30-7.16 (m, 5H), 5.72 (m, 11), 5.45 (m, 1H), 4.37 (m, 1H), 4.02 (m, 1H), 3.44 (m, 1H), 2.86-2.79 (m, 3H), 2.51 (m, 2M), 2.35-2.12 (m, 3H), 1.94 (s, 1H), 1.64-1.50 (m, 5H), 1.2 (m, 3H), 1.05 (d, J=8.0 Hz, 18H); MS 476.4 (M+1).

The difluoro component in the examples below, unless otherwise indicted, can be added to the following examples in accordance with schemes 1 and 2 herein.

### EXAMPLE 1

### (5R)-5-[(1E)-3-hydroxy-difluoro-4-phenylbut-1-enyl]-1-{4[(1-methyl-1H-tetrazol-5-yl)thio]butyl}pyrrolidin-2-one

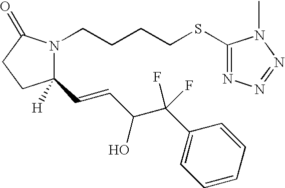

To a solution of the (5R)-1-(4-chlorobutyl)-5-[(1E)-3-hydroxy-4-phenylbut-1-enyl]pyrrolidin-2-one (50.0 mg, 0.155 mmol) in DMF (0.5 ml) wae added 5-mercapto-1-methyltetrazole sodium salt, and a catalytic amount of nBu₄NI. The mixture is heated to 50° C. overnight. The reaction is cooled to room temperature and water (5 ml) is added. The aqueous phase is extracted with AcOEt (4×10 ml), the organic phases are washed with water (2 ml), brine (2 ml), dried on MgSO₄, filtered and the solvent is removed under reduced pressure. The residual oil is purified by flash column-chrornatography on silica gel (eluent AcOEt 2: Hexanes 3) to provide both diastereoisomers of (5R)-5-[(1E)-3-hydroxy-4-difluoro-4-phenylbut-1-enyl]-1-{4-[(1-methyl-1H-tetrazol-5-yl) thio]butyl}pyrrolidin-2-one.

### EXAMPLE 2

### 4-{(2R)-2-[(1E3-hydroxy-4-difluoro-4-phenylbut-1-enyl]-5-oxopyrrolidin-1-yl}butyl thiocyanate

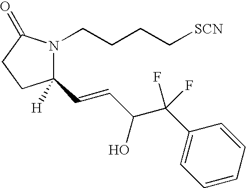

To a solution of the (5R)-1-(4-chlorobutyl)-5-[(1E)-3-hydroxy-4-phenylbut-1-enyl]pyrrolidin-2-one (50.0 mg, 0.155 mmol) in DMP (1 ml) are added potassium thiocyanate (150.7 mg, 1.55 mmol), and a catalytic amount of nBu₄NI. The mixture is heated to 50° C. overnight. The reaction is cooled to room temperature and water (5 ml) is added. The aqueous phase is extracted with AcOEt (4×10 ml), the organic phases are washed with water (2 ml), brine (2 ml), dried on MgSO₄, filtered and the solvent is removed under reduced pressure. The residual oil is purified by flash column-chromatography on silica gel (eluent Acetone 4: Toluene 6) to provide both diastereoisomers of 4-{(2R)-2-[(1E)-3-hydroxy-4-difluoro-4-phenylbut-1-enyl]-5-oxopyrrolidin-1-yl}butyl thiocyanate.

### EXAMPLE 3

### 3-[4-{(2R)-2-[(1E)-3-hydroxy-4-difluoro-4-phenylbut-1-enyl]-5-oxopyrrolidin-1-yl}butyl)thio]propanoic Acid

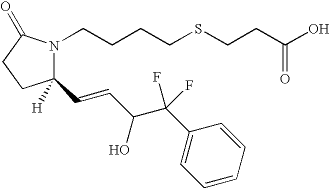

### Step A: methyl 3-[4-{(2R)-2-[(1E)-3-hydroxy-4-phenylbut-1-enyl]-5-oxopyrrolidin-1-yl}butyl)thio]propanoate

To a solution of the (5R)-1-(4-chlorobutyl)-5-[(1E)-3-hydroxy-4-phenylbut-1-enyl]pyrrolidine-2-one (50.0 mg, 0.155 mmol) in DMF (0.8 ml) are added 3-mercaptopropanoic acid methyl ester (93.0 mg, 0.755 mmol), a catalytic amount of nBu₄NI and then dropwise 1M MeONa (0.62 ml, 0.62 mmol). The mixture is heated to 80° C. for 24 h. The reaction is cooled to room temperature and water (6 ml) is added. The aqueous phase is extracted with AcOEt (4×10 ml), the organic phases are washed with water (2 ml), brine (2 ml), dried on MgSO₄, filtered and the solvent is removed under reduced pressure. The residual oil is purified by flash column-chromatography on silica gel (eluent Acetone 4: Toluene 6) to provide both diastereoisomers of methyl 3-[4-{(2R-2-[(1E)-3-hydroxy-4-phenylbut-1-enyl]-5-oxopyrrolidin-1-yl}butyl)thio]propanoate.

### Step B: 3-[4-{(2R)-2-[(1E)-3-hydroxy-4-phenylbut-1-enyl]-5-oxopyrrolidin-1-yl}butyl)thio]propanoic Acid

To a solution of methyl 3-[4-{(2R)-2-[(1E)-3-hydroxy-4-phenylbut-1-enyl]-5-oxopyrrolidin-1-yl}butyl)thio]propanoate (19.6 mg, 0.0484 mmol) in MeOH/THF (1:1)(2 ml) is added a solution of 1N LiOH (0.051 ml, 0.051 mmol) at 0° C. The reaction mixture is stirred overnight at room temperature. 0.5N HCl (4 ml) is added, the aqueous phase is extracted with CH₂Cl₂ (4×10 ml), the organic phases are washed with brine (2 ml), dried on MgSO₄, filtered and the solvent is removed under reduced pressure. The residual oil is purified by flash column-chromatography on silica gel (gradient CH₂Cl₂: MeOH: AcOH (100:0:0) to (95:5:0.5)) to provide both diastereoisomers of 3-[4-{(2R)-2-[(1E)-3-hydroxy-4-difluoro-4-phenylbut-1-enyl]-5-oxopyrrolidin-1-yl}butyl)thio]propanoic acid.

### EXAMPLE 4

### [4{(2R)-2-[(1E)-3-hydroxy-4-difluoro-4-phenylbut-1-enyl]-5-oxopyrrolidin-1-yl}butyl)thio]methanesulfonic Acid

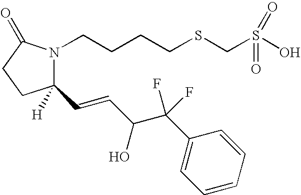

To a solution of the (5R)-5-[(ME)-3-hydroxyphenylbut-1-enyl]-1-{4-[(triisopropylsilyl)thio]butyl}pyrrolidin-2-one (39.3 mg, 0.083 mmol) in THF (1 ml) are added sodium bromomethanesulfonate (32.6 mg, 0.165 mmol) and then dropwise 1M nBu₄NF (0.25 ml, 0.25 mmol). The mixture was heated to 50° C. for 1 h. The reaction is cooled to room temperature and 1N HCl (2 ml) is added. The aqueous phase is extracted with Et₂O (4×10 ml), the organic phases are washed with 1N HCl (2 ml), brine (2 ml), dried on Na₂SO₄, filtered and the solvent is removed under reduced pressure. The residual oil is purified by flash column-chromatography on silica gel (eluent CH₂Cl₂ 95: MeOH 5:AcOH 0.5) to provide both diastereoisomers of methyl [({4-(2R)-2-[(1E)-3-hydroxydifluoro-4-phenylbut-1-enyl]-5-oxopyrrolidin-1-yl}butyl)thio]methanesulfonic acid.

### EXAMPLE 5

### (5R-5-[(1E)-3-hydroxy-4-difluoro-4-phenylbut-1-enyl]-1-[4-(methylsulfonyl)butyl]-pyrrolidin-2-one

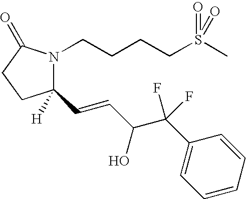

### Step A: (5R)-5-[(1E)-3-hydroxy-4-phenylbut-1-enyl[4-(methylthio)butyl]pyrrolidin-2-one

To a solution of the (5R)-5-[(1E)-3-hydroxy-4-phenylbut-1-enyl]-1-{4-[(triisopropylsilyl)thio]butyl}pyrrolidin-2-one (46.2 mg, 0.097 mmol) in THF (1 ml) were added methyliodide (17.6 mg, 0.126 mmol) and then dropwise 1M nBu₄NF (0.116 ml, 0.116 mmol) at −78° C. The mixture is then stirred at room temperature for 1 h. NH4Cl half saturated (2 ml) is added. The aqueous phase is extracted with AcOEt (5×8 ml), the organic phases are washed with brine (2 ml), dried on Na₂SO₄, filtered and the solvent is removed under reduced pressure. The residual oil is purified by flash column-chromatography on silica gel (eluent Acetone 4: Toluene 60) to provide (5R)-5-[(1E)-3-hydroxy-4-difluoro-4-phenylbut-1-enyl]-1-[4-(methylthio)butyl]-pyrrolidin-2-one.

### Step B: (5R)-5-[(1E)-3-hydroxy-4-phenylbut-1-enyl[4-(methylsulfonyl)butyl]-pyrrolidin-2-one

To a solution of (5R)-5-[(1E)-3-hydroxy-4-phenylbut-1-enyl]-1-[4-(methylthio)butyl]pyrrolidin-2-one (31.2 mg, 0.093 mmol) in CH₂Cl₂: MeOH:H₂O (7:2:1)(5 ml) was added portionwise Oxone® (172.9 mg, 0.281 mmol) at 0° C. for 10 min., and 4 h at room temperature. 5% solution of NaHSO₃ (2 ml) is added. The aqueous phase is extracted with CH₂Cl₂ (4×10 ml), the organic phases are washed with water (5 ml), brine (2 ml), dried on MgSO₄, filtered and the solvent is removed under reduced pressure. The residual oil is purified by flash column-chromatography on silica gel (eluent Acetone 7: Toluene 30) to provide (5R)-5-[(1E)-3-hydroxy-4-difluoro-4-phenylbut-1-enyl]-1-[4-(methylsulfonyl)butyl]pyrrolidin-2-one.

### EXAMPLE 6

### [4-{(2R)-2-[(1E)-3-hydroxy-4-difluoro-4-phenylbut-1-enyl]-5-oxopyrrolidin-1-yl}butyl)thio]acetic Acid

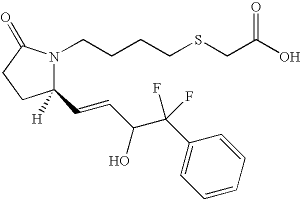

### Step A: methyl ({[4-{(2R)-2-({[tert-butyl(dimethyl)silyl]oxy}methyl)-5-o xopyrrolidin-1-yl]butyl}thio)acetate

To a solution of (5R)-(tert-butyl-dimethyl-silanyloxymethyl)-pyrrolidin-2-one (Tetrahedron: Asymmetry, 1996, 7, 2113) (1.5 g, 6.55 mmol) in 30 ml DMF is added NaH 95% (173.0 mg, 7.20 mmol) in one portion and the mixture is heated at 50° C. for 30 min. Then 4-bromo-1-chlorobutane (1.347 g, 7.86 mmol) and a catalytic amount of nBu₄NI are added and the mixture is stirred at 50° C. for 1 h. The reaction is cooled to room temperature and methyl thioglycolate (1.39 g, 13.1 mmol), then dropwise addition of 4.9N MeONa (2.4 ml, 11.79 mmol). The mixture is stirred overnight at room temperature and water (150 ml) is added. The aqueous phase is extracted with AcOEt (4×150 ml), the organic phases are washed with water (200 ml), brine (100 ml), dried on MgSO₄, filtered and the solvent is removed under reduced pressure. The residual oil is purified by flash column-chromatography on silica gel (eluent AcOEt 1: Hexanes 1) to provide methyl ({[4-{(2R)-2-({[tert-butyl(dimethyl)silyl]oxy}methyl)-5-oxopyrrolidin-1-yl]butyl}thio)acetate.

### Step B: methyl ({4-[(2R)-2-(hydroxymethyl)-5-oxopyrrolidin-1-yl]butyl}thio)acetate

To a solution of methyl ({[4{(2R)-2-({[tert-butyl(dimethyl)silyl]oxy}methyl)₅-oxopyrrolidin-1-yl]butyl}thio)acetate (571 mg, 1.47 mmol) in CH₂Cl₂ (8 ml) in a Teflon Erlenmeyer at 0° C. is added dropwise HF-pyridine complex (0.8 ml), and the solution is allowed to reach room temperature, and is stirred for 1.5 h. Water (20 ml) and 1N HCl (1 ml) were added to the reaction mixture. The aqueous phase is extracted with CH₂Cl₂ (4×30 ml), the organic phases is washed with brine (20 ml), dried on MgSO₄, filtered and the solvent is removed under reduced pressure to provide methyl ({4-[(2R)-2-(hydroxymethyl)-5-oxopyrrolidin-1-yl]butyl}thio)acetate.

### Step C: Methyl ({4-[(2R)-2-formyl-5-oxopyrrolidin-1-yl]butyl}thio)acetate

To a solution of methyl ({4-[(2R)-2-(hydroxymethyl)-5-oxopyrrolidin-1-yl]butyl}thio)acetate (634.5 mg, 2.30 mmol) in CH₂Cl₂ (15 ml) is added Dess-Martin periodinane (975 mg, 1.5 mmol) portionwise over 40 min at room temperature. After 1 h, the solvent is removed under reduced pressure, and the residue triturated with Et₂O (3×5 ml), filtered on a Celite plug, and the solvent removed. Methyl ({4-[(2R)-2-formyl-5-oxopyrrolidin-1-yl]butyl}thio)acetate is obtained

### Step D: methyl [(4-{(5R-2-oxo-5-[(1E)-3-oxo-4-phenylbut-1-enyl]pyrrolidin-1-yl}butyl)thio]acetate

To a solution of (3-phenyl-2-oxo-propyl)-phosphonic acid dimethyl ester (264 mg, 1.09 mmol) in DME (5 ml) at 0° C. is added portionwise NaH 95% (26 mg, 1.09 mmol), and the mixture is stirred 20 min at 0° C. Then a solution of methyl ({1[(2R)-2-formyl-5-oxopyrrolidin-1-yl]butyl}thio) (270 mg, 0.99 mmol) in DME (2 ml) is added dropwise and the reaction mixture is allowed to reach room temperature, and stirred overnight. A half-saturated solution of NH₄Cl (5 ml) is added and the aqueous phase is extracted with AcOEt (4×10 ml); the organic phases is washed with water (10 ml), brine (10 ml), dried on MgSO₄, filtered and the solvent is removed under reduced pressure. The residual oil is purified by flash column-chromatography on silica gel (eluent AcOEt) to provide methyl [(4-{(5R)-2-oxo-5-[(1E)-3-oxo-4-phenylbut-1-enyl]pyrrolidin-1-yl}butyl)thio]acetate.

### Step E: methyl [(4-{(2R)-2-[(1E)-3-hydroxy-4-phenylbut-1-enyl]-5-pyrrolidin-1-yl}butyl)thio]acetate

To a solution of methyl [(4-{(5R)-2-oxo-5-[(1E)-3-oxo-4-phenylbut-1-enyl]pyrrolidin-1-yl}butyl)thio]acetate (295.6 mg, 0.75 mmol) in MeOH (5 ml) at −20° C. was added portionwise NaBH₄ (27.6 mg, 1.2 mmol). The mixture is stirred at −20° C. for 1 h, and the solvent is removed under reduced pressure. The residue is dissolved in a mixture of water (5 ml) and 1N HCl (1 ml), the aqueous phase is extracted with AcOEt (3×15 ml); the organic phases is washed with water (5 ml), brine (5 ml), dried on MgSO₄, filtered and the solvent is removed under reduced pressure. The residual oil is purified by flash column-chromatography on silica gel (eluent Acetone 4: Toluene 6) to provide both diastereoisomers of methyl [(4-{(2R)-2-[(1E)-3-hydroxy-4-phenylbut-1-enyl]-5-pyrrolidin-1-yl}butyl)thio]acetate.

### Step F: [4-{(2R)-2-[(1E)-3-hydroxy-4-phenylbut-1-enyl]-5-oxopyrrolidin-1-yl}butyl)thio]acetic Acid

To a solution of methyl [(4-{(2R)-2-[(1E)-3-hydroxy-4-phenylbut-1-enyl]-5-pyrrolidin-1-yl}butyl)thio]acetate (90.0 mg, 0.23 mmol) in MeOH/TEEF (1:2)(5 ml) is added a solution of 1N LiOH (0.46 ml, 0.46 mmol) at 0° C. The reaction mixture is stirred 4 h at room temperature. 1N HCl (3 ml) is added, the aqueous phase is extracted with CH₂Cl₂ (4×10 ml), the organic phases are washed with brine (2 ml), dried on MgSO₄, filtered and the solvent is removed under reduced pressure. The residual oil is purified by flash column-chromatography on silica gel (gradient CH₂Cl₂: MeOH: AcOH (100:0:0) to (94:6:0.5)) to provide both diastereoisomers of [4-{(2R)-2-[(1E)-3-hydroxy-4-difluoro-4-phenylbut-1-enyl]-5-oxopyrrolidin-1-yl}butyl)thio]-acetic acid.

### EXAMPLE 7

### (5R)-5[(1E)-4,4-difluoro-3-hydroxy-4-phenylbut-1-enyl]-1-[6-(1H-tetraazol-5-yl)hexyl]pyrrolidin-2-one

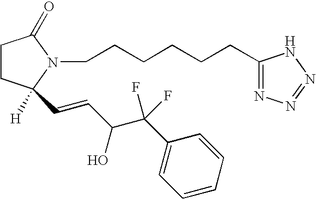

### Step A: 7-{2-Oxo-5R-[3-hydroxy-4,4-difluoro-4-phenyl)-but-1-enyl]-pyrrolidin-1-yl}-heptanenitrile

To a solution of dimethyl 3,3-difluoro-2-oxo-3-phenyl-propylphosphonate (405 mg, 1.46 mmole) in THF (5 ml) is added NaH (36.8 mg, 1.53 mmole) at 0° C., and the reaction mixture is stirred for 30 min at 0° C. A solution of 7-(2R-formyl-5-oxo-pyrrolidin-1-yl)-heptanenitrile (325 mg, 1.46 mmole) in THF (5 ml) is the addes to the above mixture. After 30 min at 0° C., the mixture is allowed to reach rt for 2 h.

Addition of a saturated solution of ammonium chloride (5 ml), extraction with AcOEt (3×15 ml); the organic phases are washed with brine, dried on Na₂SO₄ and the solvent is removed. The residue is purified by a silica gel flash chromatography (70:30 AcOEt:Hexanes) to give 7-{2-Oxo-5R-[3-oxo-4,4difluoro-4-phenyl)-but-1-enyl]-pyrrolidin-1-yl}heptanenitrile as a oil. This oil is immediately dissolved in MeOH (5 ml), and CeCl₃.7H₂O (223 mg, 0.6 mmole) is added at −20° C. NaBH (36.5 mg, 0.96 mmole) is added portionwise, and the mixture is allowed to reach rt. HCl 1N (1 ml) is added, solvent is removed in vacuo. The residue is dissolved in water (5 ml), extracted with AcOEt (3×10 ml) and the organic phases are washed with brine, dried on Na₂SO₄ and solvent removed. Purification by flash silica gel chromatography (30:70 Acetone:Toluene) to give 7-{2-Oxo-5R-[3-hydroxy-4,4-difluoro-4-phenyl)-but-1-enyl]-pyrrolidin-1-yl}-heptanenitrile as a oil. ¹H NMR (CDCl₃) δ 7.48-7.3 (m, 5H), 5.65-5.61 (m, 1), 5.57-5.53 (m, 1 h), 4.5 (m, 1H), 3.48 (m, 1H), 3.35 (m, 1H), 2.68 (m, 1H), 2.32-2.20 (m, 5H), 2.10 (m, 1H), 1.55-1.15 (m, 8H); MS 377.3 (M+1).

Step B:

To 7-{2-Oxo-5R-[3-hydroxy-4,4-difluoro-4-phenyl)-but-1-enyl]-pyrrolidin-1-yl}-heptanenitrile (193.5 mg, 0.51 mmole) is added Tributylstannylazide (512 mg, 1.54 mmole) and the mixture is heated at 100° C. for 8 h. The reaction mixture is dissolved in AcOEt and worked-up with a mixture of 1:1 (1N HCl: 5% KF). The aqueous phase is extracted by AcOEt (3×10 ml), and the organic phases are washed with brine, dried on Na₂SO₄ and solvent removed. Purification by silica gel filtration, gradient (CH₂Cl₂: MeOH: 0.1% HCOOH) from (100:0) to (94:6) to afford (5R)-5[(1E)-4,4-difluoro-3-hydroxy-4-phenylbut-1-enyl]-1-[6-(1H-tetraazol-5-yl)hexyl]pyrrolidin-2-one as an oil. ¹H NMR (CDCl₃) δ 7.5-7.35 (m, 5H), 5.7 (m, 1H), 5.6 (m, 1H), 4.6 (m, 1H), 4.05 (m, H), 3.9 (m, 2H), 2.75 (m, 1E), 2.4 (m, 2H), 2.2 (m, 1H), 1.80-1.6 (m, 3H), 1.4-1.05 (m, 6H); MS 420.3 (M+1), 418.3 (M−1).

Examples 8-11 are prepared in accordance with Scheme 4.

### EXAMPLE 8

### isopropyl 7-{(2R)-2-[(1E,3R)-4,4-difluoro-3-hydroxy-4-phenylbut-1-enyl]-5-oxopyrrolidin-1-yl}heptanoate (8a)

### Step A (5R)-5-(hydroxymethyl)-1-(4-methoxybenzyl)pyrrolidin-2-one (1a)

The preparation of 1a was carried out according to the literature procedure (see: Tetrahedron 1994, 6221)

### Step B (5R)-5-[(1E)-4,4-difluoro-3-oxo-4-phenylbut-1-enyl]-1-(4-methoxybenzyl)pyrrolidin-2-one (2a)

Oxalyl chloride (2.75 mL) was added dropwise to a solution of dimethylsulfoxide (2.45 mL) in CH₂Cl₂ (60 ml) at −72° C. under a stream of N₂, and the mixture was stirred 20 min at that temperature. A solution of (5R)-5-(hydroxymethyl)-1-(4-methoxybenzyl)pyrrolidin-2-one (1a, 6.74 g) in CH₂Cl₂ (30 ml) was then added via a cannula, and the mixture was stirred at −72° C. for 20 min. Triethylamine (8.45 ml) was then added and the mixture was stirred at −72° C. for an additional 30 min and then concentrated in vacuo (to remove most of the CH₂Cl₂). The residue was diluted with a mixture of ethyl acetate and diethyl ether and then filtered. The filtrated was concentrated to give the desired product ¹H NMR (acetone-d₆) δ 9.50 (s, 1H), 7.20 (d, 2H), 6.90 (d, 2H), 4.80 (d, 1H), 4.14 (d, 1H), 4.06 (m, 1H), 3.79, (s, 3H), 2.20-2.45 (m, 3H), 2.12 (m, 1H).

To a solution of dimethyl 3,3-difluoro-2-oxo-3-phenylpropylphosphonate (U.S. Pat. No. 4,320,136 Mar. 16, 1982) (2.076 g) in TBF (17 mL) at 0° C. was added potassium tert-butoxide (963 mg) and the mixture was stirred for an additional 1 hour at 0° C. To the mixture was then added (2R)-1-(4-methoxybenzyl)-5-oxopyrrolidine-2-carboxaldehyde in THF (10 mL) via cannula and the resultant mixture stirred at room temperature 2 hours and quenched with saturated NH₄Cl. The mixture was then extracted with ethyl acetate (3×) and the organic layer was washed with water, brine, dried over Mg₂SO₄, filtered and concentrated. The residue was purified by chromatography using 20% acetone/toluene as the eluent to give the desired product 2a. ¹H NMR (400 MHz, CDCl₃): δ 7.60-7.45 (m, 5H), 7.00 (d, 2H), 6.78 (d, 2H), 6.45 (d, 1H), 4.90 (d, 1H), 4.10-4.00 (m, 1H), 3.80 (s, 3H), 3.70 (d, 1H), 3.35 (d, 1H), 2.55-2.40 (m, 2H), 2.25-2.15 (m, 1, 1H), 1.85-1.75 (m, 1H).

### Step C (5R)-5-{(1E,3R)-4,4-difluoro-3-hydroxy-4-phenylbut-1-enyl}-1-(4 methoxybenzyl)pyrrolidin-2-one (3a)

To a solution of (2a) (8.2 g, 21.2 mmol) in 80 mL CH₂Cl₂ was added (S)-CBS in toluene (10.6 mL, 10.6 mmol) and the mixture was cooled to −40° C. A solution of catechol borane (6.8 mL, 63.8 mmol) in CH₂Cl₂ (20 mL) was added dropwise and and the solution was stirred at −40° C. for one hour, and allowed to warm up to −20° C. for two hours. The reaction mixture was quenched at −20° C. with 1 N HCl and was stirred for 4 hours at room temperature. The phases were separated and the organic phase was sequentially washed with 1N HCl, H₂O, 1 N NaOH, brine and dried over Na₂SO₄, filtered and concentrated in vacuo. The crude was purified by flash chromatography using 40-50% ethyl acetate/hexanes to give the desired products as a mixture of diastereomers as a pale yellow oil. MS (M+1) 388.2. The mixture of diastereomers was further separated by preparative HPLC (ChiralPak AD®). Eluting with 30% isopropyl alcohol in hexanes first gave isomer 4. Further elution afforded the major, more polar isomer 3a.

### Step D (5R)-5-((1E,3R)-3-{[tert-butyl(dimethyl)silyl]oxy}-4,4-difluoro-4-phenylbut-1-enyl)-1-(4-methoxybenzyl)pyrrolidin-2-one (5a)

To a solution 3a (365 mg) in DMF (3 mL) at room temperature was added imidazole (139 mg) followed by TBSCl (220 mg). The mixture was stirred over the weekend and then quenched with water. The mixture was extrated with ether (3×) and washed with water, brine, dried over Na₂SO₄, filtered and concentrated in vacuo. Purified by column chromatography (50% ethyl acetate:hexane) afforded compound 5a. ¹HNMR (400 MH, CDCl₃): δ7.55-7.40 (m, 5H), 7.15-7.10 (m, 2H), 6.85-6.80 (m, 2H), 5.65-5.55 (m, 2H), 4.95 (2d, 1H), 4.55-4.45 (m, 1H), 3.90-3.85 (m, 1H), 3.80 (s, 3H), 3.60 (2d, 1H), 2.50-2.30 (m, 2H), 2.20-2.05 (m, 1H), 1.70-1.55 (m, 1H), 0.85 (d, 9H), 0.00 (t, 6H).

### Step E (5R)-5-((1E, 3R)-3-{[tert-butyl(dimethyl)silyl]oxy}-4,4-difluoro-4-phenylbut-1-enyl)pyrrolidin-2-one (6a)

To a solution of 5a (359 mg) in acetonitrile (20 mL) at 0° C. was added CAN (ceric(IV)ammonium nitrate) (2 g), water (2 mL) and the mixture was allowed to warm to room temperature for 4 hours. The mixture was extrated with ether (3×) and was washed with water, brine and dried over Na₂SO₄. Purification by column chromatography (50%-75%-100% ethyl acetate in hexane) afforded compound 5a. ¹H NMR (400 MHz, CDCl₃): δ 7.50-7.40 (m, 5H), 5.70-5.65 (m, 2H), 4.50-4.42 (m, 1H), 4.20-4.13 (m, 1H), 2.37-2.30 (m, 3H), 1.80-1.70 (m, 1H), 0.87 (s, 9H), −0.05 (d, 6H).

### Step F

### isopropyl 7-{3-[(2R-2-((1E,3R)-3-{[tert-butyl(dimethyl)silyl]oxy}-4,4-difluoro-4-phenylbut-1-enyl)-5-oxopyrrolidin-1-yl]heptanoate (7a)

To a solution of 5a (314 mg) in DMF (dimethyl formamide −5 mL) was added NaH 60% (36.4 mg) and the mixture was stirred at room temperature for 1 h until gas evolution was ceased. Iso-propyl 7-bromoheptanoate (415 mg) and a crystal of NaI was added. The mixture was heated to 90° C. for 6 h. After cooling to room temperature, the mixture was quenched with saturated NH₄Cl and extracted with diethyl ether (3×). The organic layer was washed with water, brine and dried over Na₂SO₄. The crude was purified by flash chromatography. Eluting with 50-60% ethyl acetate in hexanes gave the desired product 6a. ¹H NMR (400 MHz, acetone-d₆) δ 7.50 (m, 5H), 7.73, m, 2H), 4.96 (m, 1H), 4.70 (m, 1H), 4.18 (m, 1H), 3.44 (m, 1H), 2.74 (m, 1H), 2.30-2.18 (m, 5H), 1.70-1.20 (m, 9H), 1.20 (d, 6H), 0.87 (s, 9H), 0.05 (s, 3H) and −0.01 (s, 3H).

### Step G

The Title Compound 8a

To a solution of 7a (350 mg) in TUF (tetrahydrofuran-5 mL) was added TBAF (1M in THF, 1.3 mL) and the mixture was stirred at room temperature for 30 min. The solution was then diluted with ethyl acetate and washed with water (4×) and brine. After drying over Na2SO4, the organic layer was filtered and concentrated in vacuo to give the desired title compoun d 8a. ¹H NMR (400 MHz, acetone-d₆) δ 7.56-7.47 (m, 51), 5.79-5.65 (m, 2), 5.05 (bs, 1H, OH), 4.95 (m, 1H), 4.66 (m, 1), 4.12 (m, 1H), 3.30 (m, 1H), 2.70 (m, 1H), 2.30-2.15 (m, 5H), 1.70-1.20 (m, 9H), 1.20 (d, 6E).

### EXAMPLE 9

### 7-{(2R)-2-[(1E,3R)-4,4-difluoro-3-hydroxy-4-phenylbut-1-enyl]-5-oxopyrrolidin-1-yl}heptanoic Acid (9a)

A mixture of compound 8a (94 mg) and LiOH (0.25 mL, 1M) in MeOH (1 mL) was stirred at room temperature overight and concentrated in vacuo. The residue was co-evaporated with MeOH three times and the resudue was washed with diethyl ether three times (to remove trace of unhygolysed ester). After neutralizing with 1N HCl, the mixture was extracted with ethyl acetate (3×). The organic layer was washed with water and brine, dried over Na₂SO₄ and filtered. The filtrate was concentrated to give the title compound 9a. MS (−ESI): m/z 394.3 (M−1)⁻.

### EXAMPLE 10

### Isopropyl 7-{(2S)-2-[(3R)-4,4-difluoro-3-hydroxy-4-phenylbutyl]-5-oxopyrrolidin-1-yl}heptanoate (10a)

To a solution of 8a (77 mg) in degassed MeOH was added Pd/C (12 mg, 10%) and the mixture was purged with hydrogen three times and then stirred under H₂ for 4.5 h. After purging with nitrogen, the mixture was filtered through a celite pad and the filtrate was concentrated to give the desired title compound 10a. ¹H NMR (400 MHz, acetone-dr) δ 7.57-7.47 (m, 5H), 4.95 (m, 1H), 4.73 (d, 1H, OH), 4.06 (m, 1H), 3.65 (m, 1H), 3.60 (m, 1H), 2.90 (m, 1H), 2.25 (t, 2H), 2.25-2.10 (m, 3H), 1.90 (m, 1H), 1.70-1.20 (m, 12H), 1.20 (d, 6H).

### EXAMPLE 11

### 7-{(2S)-2-[(3R)-4,4 difluoro-3-hydroxy-4-phenylbutyl]-5-oxopyrrolidin-1-yl}heptanoic acid (11a)

A mixture of compound 10a (30 mg) and LiOH (0.8 mL, 1M) in MeOH (2.5 mL) was stirred at room temperature for three days and neutralized with 1N HCl. The MeOH was removed in vacuo and the residue extracted with ethyl acetate (3×). The organic layer was washed with water and brine, dried over Na₂SO₄ and filtered. The filtrated was concentrated to give the title compound 11a. ¹H NMR (400 MHz, acetone-d₆) δ 7.57-7.47 (m, 5H), 4.80 (br s, 1H, OH), 4.06 (m, 1H), 3.68 (m, 1H), 3.52 (m, 1H), 2.91 (m, 1H), 2.28 (t, 2H), 2.25-2.10 (m, 3H), 1.90 (m, 1H), 1.70-1.20 (m, 12H).

I. Effects of an EP4 Agonist on Intraocular Pressure (IOP) in Rabbits and Monkeys.

Animals

Drug-naïve, male Dutch Belted rabbits and female cynomolgus monkeys are used in this study. Animal care and treatment in this investigation are in compliance with guidelines by the National Institute of Health (NH) and the Association for Research in Vision and Ophthalmology (ARVO) resolution in the use of animals for research. All experimental procedures str approved by the Institutional Animal Care and Use Committee of Merck and Company.

Drug Preparation and Administration

Drug concentrations are expressed in terms of the active ingredient (base). The compounds of this invention are dissolved in physiological saline at 0.01, 0.001, 0.0001% for rabbit study and 0.05, 0.005% for monkey studies. Drug or vehicle aliquots (25 ul) are administered topically unilaterally or bilaterally. In unilateral applications, the contralateral eyes receive an equal volume of saline. Proparacaine (0.5%) is applied to the cornea prior to tonometry to minimize discomfort. Intraocular pressure (IOP) is recorded using a pneumatic tonometer (Alcon Applanation Pneumatonograph) or equivalent.

Statistical Analysis

The results are expressed as the changes in IOP from the basal level measured just prior to administration of drug or vehicle and represent the mean, plus or minus standard deviation. Statistical comparisons are made using the Student's t-test for non-paired data between responses of drug-treated and vehicle-treated animals and for paired data between ipsilateral and contralateral eyes at comparable time intervals. The significance of the date is also determined as the difference from the “t-0” value using Dunnett's “t” test. Asterisks represent a significance level of p<0.05.

A. Intraocular Pressure Measurement in Rabbits

Male Dutch Belted rabbits weighing 2.5-4.0 kg are maintained on a 12-hour light/dark cycle and rabbit chow. All experiments are performed at the same time of day to minimize variability related to diurnal rhythm. IOP is measured before treatment then the compounds of this invention or vehicle are instilled (one drop of 25 ul) into one or both eyes and IOP is measured at 30, 60, 120, 180, 240, 300, and 360 minutes after instillation. In some cases, equal number of animals treated bilaterally with vehicle only are evaluated and compared to drug treated animals as parallel controls.

B. Intraocular Pressure Measurements in Monkeys.

Unilateral ocular hypertension of the right eye is induced in female cynomolgus monkeys weighing between 2 and 3 kg by photocoagulation of the trabecular meshwork with an argon laser system (Coherent NOVUS 2000, Palo Alto, USA) using the method of Lee at al. (1985). The prolonged increase in intraocular pressure (IOP) results in changes to the optic nerve head that are similar to those found in glaucoma patients.

For IOP measurements, the monkeys are kept in a sitting position in restraint chairs for the duration of the experiment. Animals are lightly anesthetized by the intramuscular injection of ketamine hydrochloride (3-5 mg/kg) approximately five minutes before each IOP measurement and one drop of 0.5% proparacaine was instilled prior to recording IOP. IOP is measured using a pneumatic tonometer (Alcon Applanation Tonometer) or a Digilab pneumatonometer (Bio-Rad Ophthalmic Division, Cambridge, Mass., USA).

IOP is measured before treatment and generally at 30, 60, 124, 180, 300, and 360 minutes after treatment. Baseline values are also obtained at these time points generally two or three days prior to treatment. Treatment consists of instilling one drop of 25 ul of the compounds of this invention (0.05 and 0.005%) or vehicle (saline). At least one-week washout period is employed before testing on the same animal. The normotensive (contralateral to the hypertensive) eye is treated in an exactly similar manner to the hypertensive eye. IOP measurements for both eyes are compared to the corresponding baseline values at the same time point. Results are expressed as mean plus-or-minus standard deviation in mm Hg. The activity range of the compounds of this invention for ocular use is between 0.01 and 100,000 nM.

By way of example, Compound 7 was a high affinity ligand at the EP4 receptor with a binding affinity in the range of 0.2-2 nM. It was also highly selective against other prostanoid receptors with binding affinities greater than 2 μM. In a PanLab screening against more than 80 receptors and enzymes, this compound displayed no significant activities at concentrations greater than 10 μM. The compound was a full agonist at the EP4 receptor with an EC₅₀ of 0.2-10 nM in a number of cell based functional assays using standard methods for determining EP4 functional agonist Compound 7 also had good oral bioavailability and terminal elimination half life in rats which are not known for prostaglandin analogs. This unique property allows for convenient oral dosing regimes for study EP4 agonism in in vivo models.

1. A method for treating ocular hypertension or glaucoma comprising administration to a patient in need of such treatment a therapeutically effective amount of a compound of formula I: 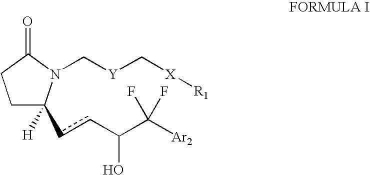
or a pharmaceutically acceptable salt, enantiomer, diastereomer, prodrug or mixture thereof, wherein,
X is (CH₂)ₙ, O or S;
Y represents (C(R^{b})₂)ₙ, triple bond, 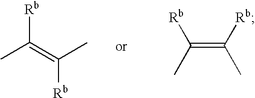
R₁ represents hydroxy, CN, CHO, NHSO₂R₆, CONHSO₂R₆, CON(R₆)₂ hydroxymethylketone, (CH₂)ₚCO₂R₆, (CH₂)ₙSO₃R₆, C₁₋₄ alkoxy, or (CH₂)ₙC₅₋₁₀heterocyclyl, said heterocyclyl unsubstituted or substituted with 1 to 3 groups of Rₐ and optionally containing an acidic hydroxyl group, with the proviso that when X is a bond R₁ is not (CH₂)ₚCO₂R₆, C₁₋₄ alkoxy, —(CH₂)ₙNR₆R₇, CHO, NHSO₂R₆, CONHSO₂R₆, CON(R₆)₂, or hydroxymethylketone;
R² and R³ independently represents hydrogen, or C₁₋₄ alkyl;
R₆ and R₇ independently represents hydrogen, or C₁₋₆ alkyl, C₃₋₁₀ cyclcoalkyl, (CH₂)ₚC₆₋₁₀aryl, (CH₂)ₚC₅₋₁₀heterocyclyl, CR²R³OC(O)OC₃₋₁₀cyclalkyl or CR²R³OC(O)O C₁₋₁₀alkyl;
Ar₂ independently represent (CH₂)ₘC₆₋₁₀aryl, (CH₂)ₘC₅₋₁₀heteroaryl, (CH₂)ₘC₃₋₁₀ heterocycloalkyl, (CH₂)ₘC₃₋₈ cycloalkyl said cycloalkyl, heterocycloalkyl, aryl or heteroaryl unsubstituted or substituted with 1-3 groups of Rₐ;
Rₐ represents C₁₋₆ alkoxy, C₁₋₆ alkyl, CF₃, nitro, amino, cyano, C₁₋₆ alkylamino, or halogen;
R^{b} independently represents H, halogen, C₁₋₆ alkyl, C₃₋₆ cylcoalkyl or 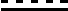
represents a double or single bond;
p represents 1-3;
n represents 0-4; and
m represents 0-8.

2. The method according to claim 1 wherein R₁ is CN, (CH₂)ₙC₅₋₁₀heterocyclyl, (CH₂)ₚCO₂R₆ or (CH₂)ₙSO₃R₆, said heterocyclyl unsubstituted or substituted with 1 to 3 groups of Rₐ and all other variables are as originally described.

3. The method according to claim 2 wherein X and Y are (CH₂)ₙ,.

4. The method according to claim 1 wherein Y is a double bond as described by 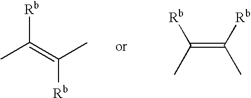
and all other variables are as originally described.

5. The method according to claim 1 wherein R₁ is (CH₂)ₙC₅₋₁₀heterocyclyl, said heterocyclyl unsubstituted or substituted with 1 to 3 groups of Rₐ, X is (CH₂)ₙ, and Y is (CH₂)ₙ or C(halo)₂.

6. The method according to claim 1 wherein R₁ is (CH₂)ₚCO₂R₆, X is (CH₂)ₙ, and Y is (CH₂)ₙ.

7. The method according to claim 1 wherein Ar₂ is (CH₂)ₘC₆₋₁₀aryl, said aryl unsubstituted or substituted with 1 to 3 groups of Rₐ and all other variables are as originally described.

8. The method according to claim 1 wherein R₁ is a tetrazole unsubstituted or substituted with an Rₐ group X is (CH₂)ₙ, and Y is (CH₂)ₙ, C(halo)₂ or a double bond as described by

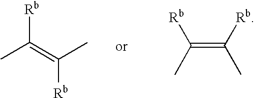

9. The method according to claim 1 wherein Ar₂ is a phenyl unsubstituted or substituted with 1 to 3 groups of Rₐ, R₁ is tetrazolyl, said tetrazolyl unsubstituted or substituted with a Rₐ group and phenyl is unsubstituted or substituted with 1-3 groups of Rₐ, and all other variables are as originally described.

10. The method according to claim wherein the compound is:
(5R)-5-[(1E)-3-hydroxy-4,4-difluoro-4-phenylbut-1-enyl]-1-{4-[(1-methyl-1H-tetrazol-5-yl)]butyl}pyrrolidin-2-one,
4-{(2R)-2-[(1E)-3-hydroxy-4,4-difluoro-4-phenylbut-1-enyl]-5-oxopyrrolidin-1-yl}butyl cyanate,
3-[4-{(2R)-2-[(1E)-3-hydroxy-4,4-difluoro-4-phenylbut-1-enyl]-5-oxopyrrolidin-1-yl}butyl)]propanoic acid,
[4-{(2R)-2-[(1E)-3-hydroxy-4,4-difluoro-4-phenylbut-1-enyl]-5-oxopyrrolidin-1-yl}butyl)]methanesulfonic acid,
(5R)-5-[(1E)-3-hydroxy-4,4-fluoro-4-phenylbut-1-enyl]-1-{4-[1H-tetrazol-5-ylmethyl)butyl}pyrrolidin-2-one,
[4-{(2R)-2-[(1E)-3-hydroxy-4,4-difluoro-4-phenylbut-1-enyl]-5-oxopyrrolidin-1-yl}butyl)]acetic acid,
(5R)-5-[(1E)-3-hydroxy-4,4-difluoro-4-phenylbut-1-enyl]-1-{4-[(1-methyl-1H-tetrazol-5-yl)thio]butyl}pyrrolidin-2-one,
(5R)-5-[(1E)-3-hydroxy-4,4-difluoro-4-phenylbut-1-enyl]-1-[4-(1H-tetrazol-5-ylthio)butyl]pyrrolidin-2-one,
3-[4-{(2R)-2-[(1E)-3-hydroxy-4,4-difluoro-4-phenylbut-1-enyl]-5-oxopyrrolidin-1-yl}butyl)thio]propanoic acid,
[4-{(2R-2-[(1E)-3-hydroxy-4,4-difluoro-4-phenylbut-1-enyl]-5-oxopyrrolidin-1-yl}butyl)thio]methanesulfonic acid,
(5R)-5-[(1E)-3-hydroxy-4,4-difluoro-4-phenylbut-1-enyl]-1-[4-(methylsulfonyl)butyl]-pyrrolidin-2-one,
[4-{(2R-2-[(1E)-3-hydroxy-4,4-difluoro-4-phenylbut-1-enyl]-5-oxopyrrolidin-1-yl}butyl)thio]acetic acid,
(5R)-5[(1E)-4,4-difluoro-3-hydroxy-4-phenylbut-1-enyl]-1-[6-(1H-tetrazol-5-yl)hexyl]pyrrolidin-2-one,
7-{(2R)-2-[(1E,3R)-4,4-difluoro-3-hydroxy-4-phenylbut-1-enyl]-5-oxopyrrolidin-1-yl}heptanoic acid,
isopropyl 7-{(2R)-2-[(1E,3R)-4,4-difluoro-3-hydroxy-4-phenylbut-1-enyl]-5-oxopyrrolidin-1-yl}heptanoate,
7-{(2S)-2-[(3R)4,4-difluoro-3-hydroxy-4-phenylbutyl]-5-oxopyrrolidin-1-yl}heptanoic acid,
(5Z)-7-{(R)-2-[(1E,3R)-4,4-difluoro-3-hydroxy-4-phenylbut-1-enyl]-5-oxopyrrolidin-1-yl}hept-5-enoic acid,
isopropyl (5Z)-7-{(2R)-2-[(1E,3R)-4,4-difluoro-3-hydroxy-4-phenylbut-1-enyl]-5-oxopyrrolidin-1-yl}hept-5-enoate,
7-{(2R)-2-[(1E,3R)-4-(3-chlorophenyl)-4,4-difluoro-3-hydroxybut-1-enyl]-5-oxopyrrolidin-1-yl}heptanoic acid,
isopropyl 7-{(2R)-2-[(1E,3R)-4-(3-chlorophenyl)-4,4-difluoro-3-hydroxybut-1-enyl]-5-oxopyrrolidin-1-yl}heptanoate,
7-((2R)-2-{(1E,3R)-4,4-difluoro-3-hydroxy-4-[3-trifluoromethyl)phenyl]but-1-enyl}-5-oxopyrrolidin-1-yl)heptanoic acid,
isopropyl 7-{(2R)-2-{(1E,3R)-4,4-difluoro-3-hydroxy-4-[3-(trifluoromethyl)phenyl]but-1-enyl}-5-oxopyrrolidin-1-yl)heptanoate,
cyclopentyl 7-{(2R)-2-[(1E,3R)-4,4-difluoro-3-hydroxy-4-phenylbut-1-enyl]-5-oxopyrrolidin-1-yl}heptanoate,
7-{(2R)-2-[(1E,3R)-4,4-difluoro-3-hydroxy-3-methyl-4-phenylbut-1-enyl]-5-oxopyrrolidin-1-yl}heptanoic acid,
isopropyl 7-{(2R)-2-[(1E,3R)-4,4-difluoro-3-hydroxy-3-methyl-4-phenylbut-1-enyl]-5-oxopyirolidin-1-yl}heptanoate,
isobutyl 7-{(2R)-2-[(1E,3R)-4, fluoro-3-hydroxy-4-phenylbut-1-enyl]-5-oxopyrrolidin-1-yl}heptanoate,
cyclohexyl 7-{(2R)-2-[(1E,3R)-4,4-difluoro-3-hydroxy-4-phenylbut-1-enyl]-5-oxopyrrolidin-1-yl}heptanoate,
(5R)-5-[(1E)-(3R)-hydroxy-4,4-difluoro-4-phenylbut-1-enyl]-1-{4-[(1-methyl-1H-tetrazol-5-yl)]butyl}pyrrolidin-2-one,
4-{(2R)-2-[(1E)-(3R)-hydroxy-4,4-difluoro-4-phenylbut-1-enyl]-5-oxopyrrolidin-1-yl}butyl cyanate,
3-[4-{(2R}2-[(1E)-(3R)-hydroxy-4,4-difluoro-4-phenylbut-1-enyl]-5-oxopyrrolidin-1-yl}butyl)]propanoic acid,
[4{(2R)-2-[(1E)-(3R)-hydroxy-4,4-difluoro-4-phenylbut-1-enyl]-5-oxopyrrolidin-1-yl}butyl)]methanesulfonic acid,
(5R)-5-[(1E)-(3R)-hydroxy-4,4-difluoro-4-phenylbut-1-enyl]-1-{4-[1H-tetrazol-5-ylmethyl)butyl}pyrrolidin-2-one,
[4-{(2R)-2-[(1E)-(3R)-hydroxy-4,4-difluoro-4-phenylbut-1-enyl]-5-oxopyrrolidin-1-yl}butyl)]acetic acid,
(5R)-5-[(1E)-(3R)-hydroxy-4,4-difluoro-4-phenylbut-1-enyl]-1-{4-[(1-methyl-1H-tetrazol-5-yl)thio]butyl}pyrrolidin-2-one,
(5R)-5-[(1E)-(3R)-hydroxy-4,4-difluoro-4-phenylbut-1-enyl]-1-[4-(1H-tetrazol-5-ylthio)butyl]pyrrolidin-2-one,
3-[4-{(2R)-2-[(1E)-(3R)-hydroxy-4,4-difluoro-4-phenylbut-1-enyl]-5-oxopyrrolidin-1-yl}butyl)thio]propanoic acid,
[4-{(2R)-2-[(1E)-(3R)-hydroxy-4,4-difluoro-4-phenylbut-1-enyl]-5-oxopyrrolidin-1-yl}butyl)thio]methanesulfonic acid,
(5R)-5-[(1E)-(3R)-hydroxy-4,4-difluoro-4-phenylbut-1-enyl]-1-[4-(methylsulfonyl)butyl]-pyrrolidin-2-one,
[4{(2R)-2-[(1E)-(3R)-hydroxy-4,4-difluoro-4-phenylbut-1-enyl]-5-oxopyrrolidin-1-yl}butyl)thio]acetic acid, or
(5R)-5 [(1E)-4,4-difluoro-(3R)-hydroxy-4-phenylbut-1-enyl]-1-[6-(1H-tetrazol-5-yl)hexyl]pyrrolidin-2-one, a pharmaceutically acceptable salt, enantiomer, diastereomer, prodrug, or mixture thereof.

11. A method according to claim 1, which is administered in a topical formulation as a solution or suspension.

12. A method according to claim 1 wherein a second active ingredient belonging to the group consisting of: O-adrenergic blocking agent, parasympatho-mimetic agent, sympathomimetic agent, carbonic anhydrase inhibitor, Maxi-K channel blocker, and a prostaglandin, hypotensive lipid, neuroprotectant, and 5-HT2 receptor agonist is added to the topical formulation.

13. A method according to claim 12 wherein the O-adrenergic blocking agent is timolol, betaxolol, levobetaxolol, carteolol, or levobunolol; the parasympathomimetic agent is pilocarpine; the sympathomimetic agent is epinephrine, brimonidine, iopidine, clonidine, or para-aminoclonidine, the carbonic anhydrase inhibitor is dorzolamide, acetazolamide, metazolamide or brinzolamide; the prostaglandin is latanoprost, travaprost, unoprostone, rescula, or S1033, the hypotensive lipid is lumigan, the neuroprotectant is eliprodil, R-eliprodil or memantine; and the 5-HT2 receptor agonist is 1-(2-aminopropyl)-3-methyl-1H-imdazol-6-ol fumarate or 2-(3-chloro-6-methoxy-indazol-1-yl)-1-methyl-ethylamine.

14. A method for treating macular edema, macular degeneration, treating dry eye, increasing retinal and optic nerve head blood velocity, increasing retinal and optic nerve oxygen tension or providing a neuroprotection comprising administration to a patient in need of such treatment a pharmaceutically effective amount of a compound of formula I as recited in claim 1

15. The method according to claim 14 wherein the compound of formula I is applied as a topical formulation and an active ingredient belonging to the group consisting of β-adrenergic blocking agent, parasympatho-mimetic agent, sympathomimetic agent, carbonic anhydrase inhibitor, Maxi-K channel blocker and a prostaglandin, hypotensive lipid, neuroprotectant, and 5-HT2 receptor agonist is added to the formulation.

16. A method according to claim 15 wherein the the β-adrenergic blocking agent is timolol, betaxolol, levobetaxolol, carteolol, or levobunolol; the parasympathomimetic agent is pilocarpine; the sympathornimetic agent is epinephrine brimonidine, iopidine, clonidine, or para-aminoclonidine, the carbonic anhydrase inhibitor is dorzolamide, acetazolamide, metazolamide or brinzolamide; the prostaglandin is latanoprost, travaprost, unoprostone, rescula, or S1033, the hypotensive lipid is lumigan, the neuroprotectant is eliprodil, R-eliprodil or memantine; and the 5-HT2 receptor agonist is 1-(2-aminopropyl)-3-methyl-1H-imdazol-6-ol fumarate or 2-(3-chloro-6-methoxy-indazol-1-yl)-1-methyl-ethylamine.

17. A compound of structural formula I: 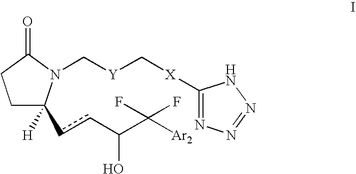
or a pharmaceutically acceptable salt, enantiomer, diastereomer, pro drug or mixture thereof, wherein
X is (CH₂)ₙ, O or S;
Y represents (C(R^{b})₂)ₙ, triple bond, 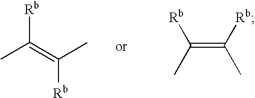
Ar₂ independently represent (CH₂)ₘC₆₋₁₀aryl, (CH₂)ₘC₅₋₁₀heteroaryl, (CH₂)ₘC₃₋₁₀heterocycloalkyl, (CH₂)ₘC₃₋₈ cycloalkyl said cycloalkyl, heterocycloalkyl, aryl or heteroaryl unsubstituted or substituted with 1-3 groups of Rₐ;
Rₐ represents C₁₋₆ alkoxy, C₁₋₆ alkyl, CF₃, nitro, amino, cyano, C₁₋₆ alkylamino, or halogen;
R_{b} independently represents H, halogen, C₁₋₆ alkyl, C₃₋₆ cylcoalkyl or 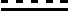
represents a double or single bond;
n represents 0-4; and
m represents 0-8.

18. The compound according to claim 17 wherein X and Y are (CH₂)ₙ, 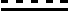
represents a double bond; and AR₂ is phenyl.

19. The compound according to claim 18 wherein X is (CH₂)ₙ and n is 1 and Y is (CH₂)ₙ and n is 3.

20. Use of a compound of formula I, as defined in any one of claims 1 to 10, or a pharmaceutically acceptable salt, enantiomer, diasteromer, prodrug, or mixture thereof, in the manufacture of a medicament for treating hypertension or glaucoma

21. A pharmaceutical composition for treating hypertension or glaucoma comprising a therapeutically effective amount of a compound of formula I, as defined in any one of claims 1 to 10, or a pharmaceutically acceptable salt, enantiomer, diasteromer, prodrug, or mixture thereof, in association with a pharmaceutically acceptable carrier.

22. A composition according to claim 21 in a form for topical administration as a solution or suspension and further comprising a second active ingredient as defined in claim 12 or 13.

23. Use of a compound of formula I, as defined in any one of claims 1 to 10, or a pharmaceutically acceptable salt, enantiomer, diasteromer, prodrug, or mixture thereof, in the manufacture of a medicament for treating macular edema, macular degeneration, dry eye, increasing retinal and optic nerve velocity, increasing retinal and optic nerve oxygen tension or providing a neuroprotection.

24. A pharmaceutical composition for treating macular edema, macular degeneration, dry eye, increasing retinal and optic nerve velocity, increasing retinal and optic nerve oxygen tension or providing a neuroprotection comprising a therapeutically effective amount of a compound of formula I, as defined in any one of claims 1 to 10, or a pharmaceutically acceptable salt, enantiomer, diasteromer, prodrug, or mixture thereof, in association with a pharmaceutically acceptable carrier.

25. A composition according to claim 24 in a form for topical administration and further comprising an active ingredient as defined in claim 15 or 16.

26. A compound of claim 17, 18 or 19 for use in medicinal therapy.
